(19) United States
(12) Patent Application Publication
(10) Pub. No.: US 2025/0262346 A1
(43) Pub. Date: Aug. 21, 2025

(54) FUNCTIONALIZED BIOMATERIALS FOR ADHESION AND INTERNAL DEVICE APPLICATIONS

(71) Applicants: Mohammad Ali DARABI, Oakland, CA (US); Peyton TEBON, Glendale, AZ (US); THE REGENTS OF THE UNIVERSITY OF CALIFORNIA, Oakland, CA (US)

(72) Inventors: Renea Sturm, Los Angeles, CA (US); George Aninwene, Los Angeles, CA (US); Mohammad Ali Darabi, Oakland, CA (US); Peyton Tebon, Oakland, CA (US); Alireza Khademhosseini, Oakland, CA (US)

(21) Appl. No.: 18/857,649

(22) PCT Filed: May 1, 2023
(86) PCT No.: PCT/US23/66448

§ 371 Date: Oct. 17, 2024

Related U.S. Application Data
(60) Provisional application No. 63/336,769, filed on Apr. 29, 2022.

(51) Int. Cl. A61L 24/04; A61L 24/00; C09J 167/02

(52) U.S. Cl. CPC A61L 24/046; A61L 24/0042; C09J 167/025

(57) ABSTRACT

The present invention relates to non-toxic, biodegradable adhesive compositions comprising the reaction product of a linear dicarboxylic acid, a saturated triol, and an aromatic amino acid. In certain embodiments, the adhesive compositions comprise the reaction product of sebacic acid terminated-poly(ethylene glycol), glycerin, and L-DOPA. The present invention also relates to methods of using the adhesive compositions for the closure of various wounds. Further, the present invention provides surgical closure devices comprising an adhesive composition.

### CROSS-REFERENCE TO RELATED APPLICATIONS

This application claims priority under 35 U.S.C. § 119 (e) to U.S. Provisional Patent Application Ser. No. 63/336,769, filed Apr. 29, 2022, the disclosure of which is incorporated herein by reference in its entirety.

### STATEMENT OF GOVERNMENT SUPPORT

This invention was made with government support under 2045366, awarded by the National Science Foundation. The government has certain rights in the invention.

### BACKGROUND OF THE INVENTION

Various tissues and lumens throughout the body frequently require repair, representing procedures as diverse as vascular anastomoses in a transplant to management of a congenital esophageal condition. The current gold standard for these repairs is often a sutured surgical closure in multiple layers to decrease risk of complications such as leak, fistula, or erosion.

The use of rapidly applied, consistent closure methods that have become standard of care in various fields, such as genitourinary or gastrointestinal surgery, are not always compatible with other procedures. In some applications, permanent staples or stents used to repair the tissue serve as a nidus for stone formation or infection.

While a variety of patches and other closure devices exist, the outcomes of their use would be greatly enhanced by a superior adhesive material that is particularly suited for internal biological applications. Thus, there is a need in the art for biocompatible adhesives that may be applied to any tissue or surface required. The present invention meets this need.

### SUMMARY OF THE INVENTION

In certain aspects, the present invention provides adhesive compositions comprising the reaction product of a linear dicarboxylic acid, a saturated triol, and an aromatic amino acid, wherein the adhesive is non-cytotoxic and biodegradable. In some embodiments, the linear dicarboxylic acid comprises the reaction product of a saturated linear dicarboxylic acid and a linear polymer. In some embodiments, the saturated linear dicarboxylic acid is one or more selected from the group consisting of: succinic acid, glutaric acid, adipic acid, pimelic acid, suberic acid, azelaic acid, sebacic acid, undecanedioic acid, dodecanedioic acid, brassylic acid, tetradecanedioic acid, pentadecanedioic acid, thapsic acid, heptadecanedioic acid, japanic acid, and phellognic acid. In some embodiments, the linear dicarboxylic acid is one or more selected from the group consisting of: succinic acid, glutaric acid, adipic acid, pimelic acid, suberic acid, azelaic acid, sebacic acid, undecanedioic acid, dodecanedioic acid, brassylic acid, tetradecanedioic acid, pentadecanedioic acid, thapsic acid, heptadecanedioic acid, japanic acid, and phellognic acid. In some embodiments, the linear polymer is one or more selected from the group consisting of poly(ethylene glycol) (PEG), poly(lactic acid) (PLA), poly(caprolactone) (PCL), poly(glycolic acid) (PGA), poly(lactic-co-glycolic acid) (PLGA), and poly(glycerol sebacate) (PGS). In some embodiments, the linear polymer is PEG, wherein the PEG comprises one or more selected from the group consisting of: PEG100, PEG200, PEG300, PEG400, PEG500, PEG600, PEG700, PEG800, PEG 900, or PEG1000.

In some embodiments, the saturated triol is one or more selected from the group consisting of glycerol, 1,2,4-butanetriol, 2-hydroxymethyl-1,3-propanediol, 1,3,5-propanetriol, 2-hydroxymethyl-1,4-butanediol, 1,2,6-hexanetriol, 1,3,6-hexanetriol, 2-hydroxymethyl-1,5-pentanediol, 3-hydroxymethyl-1,5-pentanediol, 1,2,7-heptanetriol, 1,3,7-heptanetriol, 1,4,7-heptanetriol, 2-hydroxymethyl-1,6-hexanediol, 3-hydroxymethyl-1,6-hexanediol, and 3-hydroxyethyl-1,5-pentanediol.

In some embodiments, the aromatic amino acid is of formula (I):

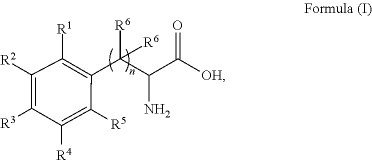

wherein R¹-R⁵ are each independently selected from the group consisting of H, D, F, Cl, Br, hydroxyl, hydroxymethyl, hydroxyethyl, methoxy, ethoxy, methyl, ethyl, amino, aminomethyl, aminoethyl, methylamino, and ethylamino;
wherein at least one of R¹-R⁵ is a hydroxyl;
wherein n=1-6; and
wherein each instance of R⁶ is independently selected from H, D, F, and Cl.

In some embodiments, the reaction product of a saturated linear dicarboxylic acid and a linear polymer comprises 40-60% saturated linear dicarboxylic acid by weight.

In some embodiments, the adhesive composition comprises 50-85% reaction product of a saturated linear dicarboxylic acid and a PEG by weight. In some embodiments, the composition comprises 10-30% saturated triol by weight. In some embodiments, composition comprises 1-5% aromatic amino acid by weight.

In some embodiments, the saturated linear dicarboxylic acid is sebacic acid, the PEG is PEG400, the saturated triol is glycerol, and the aromatic amino acid is L-DOPA. In some embodiments, the composition comprises: 50-85% reaction product of sebacic acid and PEG400 by weight, 10-30% glycerol by weight, and 1-5% L-DOPA by weight, wherein the reaction product of sebacic acid and PEG400 is 40-60% saturated linear dicarboxylic acid by weight.

In some aspects, the present invention provides self-adhesive devices comprising a flexible base and an adhesive layer comprising an adhesive composition of the invention, wherein the flexible base is non-toxic. In some embodiments, the flexible base comprises a polymer. In some embodiments, the polymer is biodegradable. In some embodiments, the biodegradable polymer is selected from the group consisting of polylactic acid (PLA), poly(L-lactic acid) (PLLA), poly(D,L-lactic acid) (PDLLA), polycaprolactone (PCL), chitosan, polyglycolic acid (PGA), poly(lactic-go-glycolic acid) (PLGA), poly(3-hydroxybuterate-co-3-hydroxyvalerate (PHBV), chitin, polyhydroxyalkanoates (PHAs), polyamides, poly(glycerol sebacate) (PGS), polydioxanone (PDS), poly(trimethylene carbonate) (PTMC), cyanophycin, PEG, polyethylene glycol diacrylate (PEGDA), poly(vinyl alcohol) (PVA), poly(lactic acid-co-caprlocactone) (PLCL), poly(ester urea) (PEU), and poly(ester urethane). In some embodiments, the polymer is PGS.

In some embodiments, the device is a biozipper. In some embodiments, the device is a biotape. In some embodiments, the adhesive layer comprises a therapeutic agent.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1, comprising FIG. 1A and FIG. 1B, depicts a perspective view of an exemplary bio-zipper surgical closure device. FIG. 1A depicts a perspective view of an exemplary bio-zipper surgical closure device placed on 3d printed model of a urethra. FIG. 1B depicts a perspective view of an exemplary bio-zipper surgical closure device.

FIG. 2 depicts a perspective view of multiple exemplary bio-zipper surgical closure device of the present invention.

FIG. 3 depicts a perspective view of an exemplary biotape surgical closure device of the present invention.

FIG. 4 depicts a perspective view of another exemplary biotape surgical closure device of the present invention.

FIG. 5 depicts a perspective view of another exemplary biotape surgical closure device of the present invention.

FIG. 6 is a flowchart depicting an exemplary method of wound closure using an exemplary bio-zipper surgical closure device of the present invention.

FIG. 7 is a flowchart depicting an exemplary method of wound closure using an exemplary bio-zipper surgical closure device of the present invention.

FIG. 8 is a flowchart depicting an exemplary method of wound closure using an exemplary biotape surgical closure device of the present invention.

FIG. 9, comprising FIG. 9A through FIG. 9C, depicts synthesis of poly(glycerol sebacate) (PGS) and the mechanical properties of PGS (Pomerantseva I. et al., 2009, Journal of Biomedical Materials Research Part A, 91A: 1038-1047; Wang Y. et al., 2002, Nature Biotech., 20:602-606). FIG. 9A depicts that PGS is synthesized by the polycondensation of glycerol and sebacic acid. FIG. 9B depicts Stress-strain curves for PGS as a function of curing time. FIG. 9C depicts Young's modulus (YM) for PGS as a function of curing time.

FIG. 10, comprising FIG. 10A and FIG. 10B, depicts representative preparation of secondary curing PGS polymer. FIG. 10A depicts representative PGS pre-polymer after 0 h at 140° C. FIG. 10B depicts representative PGS after 14 h at 140° C.

FIG. 11, comprising FIG. 11A and FIG. 11B, depicts representative PGS and its tensile data. FIG. 11A depicts representative results demonstrating the tensile strength, Young's modulus, and tensile strain at failure of PGS samples that was cured at 140° C. for 8, 14, or 20 h (error±SD, n=3, ** p≤0.01, *** p≤0.001). FIG. 11B depicts representative PGD sample.

FIG. 12, comprising FIG. 12A and FIG. 12B, depicts representative results of PGS biodegradation. FIG. 12A depicts representative images of PGS that was incubated in human plasma at 37° C. for up to 21 days. PGS examined had a 1:1 ratio of sebacic acid:glycerol (top), 1:1.5 ratio (middle), or 1:2 (bottom). FIG. 12B depicts representative quantification of the degradation of PGS as shown in FIG. 12A.

FIG. 13 depicts a schematic representation of synthesis of sebacic acid terminated polyethylene glycol (PEG) and the synthesis of L-3,4-dihydroxyphenylalanine (L-DOPA) terminated co-polymer of sebacic acid terminated PEG and polyglycerol (PGS-PEG-L-DOPA) (Zhao Y. et al., 2021, Advanced Functional Materials, 31:2008325).

FIG. 14, comprising FIG. 14A and FIG. 14B, depicts representative sebacic acid terminated PEG and ¹H NMR spectrum thereof. FIG. 14A depicts a representative ¹H NMR spectrum of sebacic acid-terminated PEG. FIG. 14B depicts a representative image of a representative sebacic acid terminated PEG.

FIG. 15, comprising FIG. 15A and FIG. 15B, depicts schematic representation of PGS-PEG-L-DOPA and polyglycerol and representative ¹H NMR spectrum thereof. FIG. 15A depicts schematic representation of PGS-PEG-L-DOPA. FIG. 15B depicts a representative ¹H NMR spectrum of PGS-PEG-L-DOPA.

FIG. 16, comprising FIG. 16A and FIG. 16B, depicts representative images of a representative PGS-PEG-L-DOPA before and after secondary curing at 140° C. for 3 days. FIG. 16A depicts a representative PGS-PEG-L-DOPA before secondary curing at 140° C. for 3 days. FIG. 16B depicts a representative PGS-PEG-L-DOPA after secondary curing at 140° C. for 3 days.

FIG. 17, comprising FIG. 17A and FIG. 17B, depicts representative experimental results of the mechanical properties of PGS and PGS-PEG (3000)-L-DOPA. FIG. 17A depicts representative experimental results of the mechanical properties of PGS, demonstrating that an increasing ratio of glycerol to sebacic acid (1:1, “1”; 1.5:1, “1.5”; and 2:1, “2”) increases PGS elasticity. FIG. 17B depicts representative experimental results of the mechanical properties of PGS-PEG (3000)-L-DOPA. High molecular weight PEG results in brittle polymers.

FIG. 18, comprising FIG. 18A through FIG. 18C, depicts representative experimental results of the mechanical properties of PGS-PEG (400)-L-DOPA. FIG. 18A depicts representative results of PGS-PEG (400)-L-DOPA with a L-DOPA loading of 1 (PGS-PEG (400)-L-DOPA (1)). FIG. 18B depicts representative results of PGS-PEG (400)-L-DOPA with a L-DOPA loading of 0.25 (PGS-PEG (400)-L-DOPA (0.25)). FIG. 18A depicts representative results of PGS-PEG (400)-L-DOPA with a L-DOPA loading of 0.006 (PGS-PEG (400)-L-DOPA (0.006)).

FIG. 19, comprising FIG. 19A through FIG. 19C, depicts representative results demonstrating cytocompatibility of PGS-PEG-L-DOPA with L-DOPA loading between 0.06 and 1. FIG. 19A depicts a schematic representation of an experimental setup used to examine polymer cytocompatibility. FIG. 19B depicts representative fluorescent images of cells exposed to various polymers. FIG. 19C depicts quantification of cell viability from FIG. 19B.

FIG. 20 depicts representative experimental results demonstrating polymer swelling in solution. PGS and PGS-PEG (400)-L-DOPA (0.25) were examined with glycerol:sebacic acid ratios of 1:1 (1), 1.5:1 (1.5), and 2:1 (2).

FIG. 21, comprising FIG. 21A through FIG. 21C, depicts representative results of lap shear adhesive strength analysis on collagen sheets. FIG. 21A depicts a schematic representation of lap shear adhesive strength analysis. FIG. 21B depicts representative experimental results comparing PGS-PEG-L-DOPA (14 hour cure PGS-PEG-L-DOPA), TISSEEL (Murphy, Biomacromolecule), and GelMA-Dopa (n=3, p<0.002). FIG. 21C depicts representative experimental results comparing PGS with a glycerol:sebacic acid ratio of 2:1 (PGS (2)) to PGS-PEG (400)-L-DOPA. Polymer loading with 0.06 L-DOPA was tested with a glycerol:sebacic acid ratio of 1.5:1 (1.5) and 2:1 (2). Polymer loading with 0.25 L-DOPA was also tested with a glycerol:sebacic acid ratio or 2:1.

FIG. 22 depicts representative results of lap shear adhesive strength analysis, as in FIG. 21C, on bladder tissue. PGS with a glycerol:sebacic acid ratio of 2:1 (PGS (2)) to PGS-PEG (400)-L-DOPA. Polymer loading with 0.06 L-DOPA was tested with a glycerol:sebacic acid ratio of 1.5:1 (1.5) and 2:1 (2). Polymer loading with 0.25 L-DOPA was also tested with a glycerol:sebacic acid ratio or 2:1.

FIG. 23, comprising FIG. 23A and FIG. 23B, depicts representative results of tensile adhesive strength analysis. FIG. 23A depicts a schematic representation of tensile adhesive strength analysis. FIG. 23B depicts representative experimental results comparing PGS with a glycerol:sebacic acid ratio of 2:1 (PGS (2)) to PGS-PEG (400)-L-DOPA. Polymer loading with 0.06 L-DOPA was tested with a glycerol:sebacic acid ratio of 1.5:1 (1.5) and 2:1 (2). Polymer loading with 0.25 L-DOPA was also tested with a glycerol:sebacic acid ratio or 2:1.

FIG. 24, comprising FIG. 24A and FIG. 24B, depicts representative results of peeling adhesive strength analysis. FIG. 24A depicts a schematic representation of peeling adhesive strength analysis. FIG. 24B depicts representative experimental results comparing PGS with a glycerol:sebacic acid ratio of 2:1 (PGS (2)) to PGS-PEG (400)-L-DOPA. Polymer loading with 0.06 L-DOPA was tested with a glycerol:sebacic acid ratio of 1.5:1 (1.5) and 2:1 (2). Polymer loading with 0.25 L-DOPA was also tested with a glycerol:sebacic acid ratio or 2:1.

FIG. 25, comprising FIG. 25A and FIG. 25B, depicts representative experimental results of a burst pressure test. FIG. 25A depicts representative images of a burst pressure test experimental setup. A 1 cm incision was cut in a porcine bladder, which was closed with a double-layer stitch closure with 3-0 VICRYL. FIG. 25B depicts representative results of the pressure test comparing a bare suture to a suture coupled with a Coseal, Tiseel, or PGS (2)-PEG-L-DOPA (0.06) patch.

FIG. 26 depicts a schematic representation of 2-layer patch comprising PGS-PEG-L-DOPA adhesive layer and PGS backbone.

FIG. 27, comprising FIG. 27A and FIG. 27B, depicts representative 2-layer patch and its burst pressure results. FIG. 27A depicts representative 2-layer patch. FIG. 27B depicts representative results demonstrating the burst pressure effect of the 2-layer patch.

### DETAILED DESCRIPTION

The present invention is based, in part, on the unexpected results that a functionalized polymer comprising L-3,4-dihydroxyphenylalanine (L-DOPA) possesses significant adhesive properties. Thus, in various embodiments, the present invention provides adhesive compositions comprising functionalized polymers suitable for biological applications including use as a sealant and as an adhesive layer of implantable patches, tapes and other closure devices. In some embodiments, the polymers are functionalized with one or more hydroxylated aromatic substituents. In some embodiments, the polymer comprises one or more dicarboxylic acids. In some embodiments, the polymer comprises one or more saturated triols. In some embodiments, the polymer is biodegradable.

In another aspect, the present invention also provides adhesive devices. In some embodiments, the adhesive device is a self-adhesive surgical closure device. In some embodiments, the self-adhesive surgical closure device comprises a flexible base and an adhesive layer comprising an adhesive composition of the present invention.

In other aspects, the present invention provides a method for wound closure using the adhesive composition or surgical closure device.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

The term “biological tissue” as used herein refers to a collection of interconnected cells and extracellular matrix that perform a similar function or functions within an organism Examples of biological tissues include, but are not limited to, connective tissue, muscle tissue, nervous tissue (of the brain, spinal cord, and nerves), epithelial tissue, organ tissue, cancer tissue, and any combination thereof. Connective tissue includes fibrous tissue like fascia, tendon, ligaments, heart valves, bone, and cartilage. Muscle tissue includes skeletal muscle tissue, smooth muscle tissue, such as esophageal, stomach, intestinal, bronchial, uterine, urethral, bladder, and blood vessel tissue, and cardiac muscle tissue. Epithelial tissue includes simple epithelial tissue, such as alveolar epithelial tissue, blood vessel endothelial tissue, and heart mesothelial tissue, and stratified epithelial tissue.

The term “derivative” refers to a molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C═N, —C(═O)O(C₁-C₄)alkyl, —C(═O)NH₂, —SO₂NH₂, —C(—NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized, and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom. As used herein, the term “cycloalkyl” refers to a mono cyclic or polycyclic non-aromatic radical, wherein each of the atoms forming the ring (i.e., skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

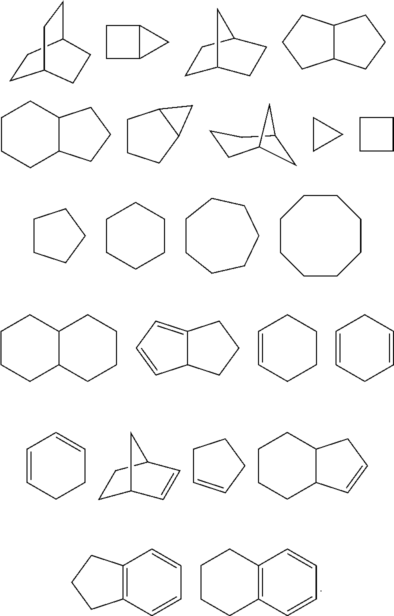

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

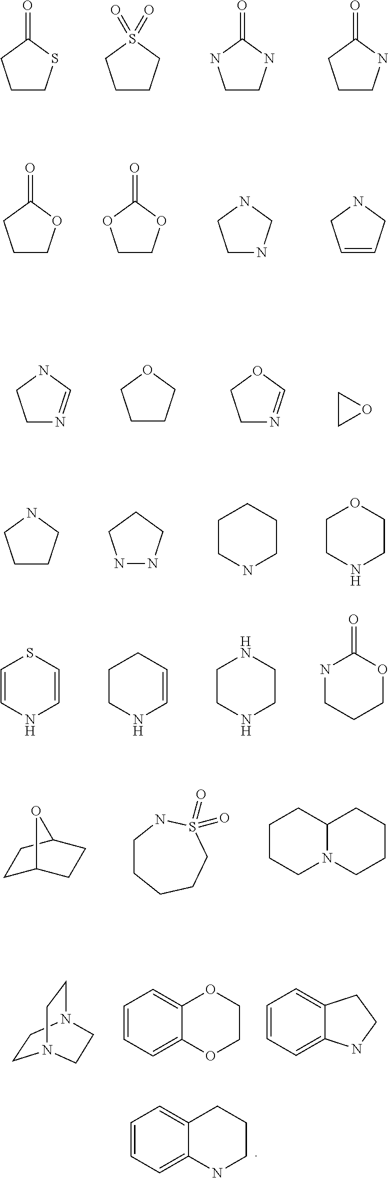

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “heteroaryl” or “heteroaromatic” refers to a heterocycle having aromatic character. A polycyclic heteroaryl may include one or more rings that are partially saturated. Examples include the following moieties:

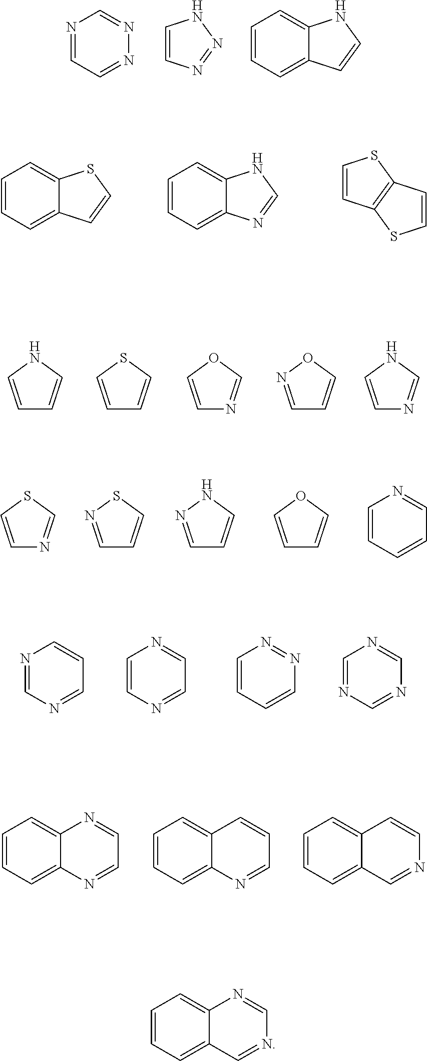

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(—O)₂alkyl, —C(—O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

The term “tautomers” are constitutional isomers of compounds that readily interconvert by a chemical process (tautomerization).

The term “isomers” or “stereoisomers” refer to compounds, which have identical chemical constitution, but differ with regard to the arrangement of the atoms or groups in space.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

The present invention relates, in part, to adhesive compositions. In some embodiments, the adhesive compositions comprise functionalized polymers. In some embodiments, the functionalized polymers comprise the reaction product of a linear dicarboxylic acid, a saturated triol, and an aromatic amino acid.

In some embodiments, one of the reagents used in preparation of the functionalized polymer is a linear dicarboxylic acid. In some embodiments, the linear dicarboxylic acid is a saturated linear dicarboxylic acid. In some embodiments, the saturated linear dicarboxylic acid is one or more selected from the group consisting of: succinic acid, glutaric acid, adipic acid, pimelic acid, suberic acid, azelaic acid, sebacic acid, undecanedioic acid, dodecanedioic acid, brassylic acid, tetradecanedioic acid, pentadecanedioic acid, thapsic acid, heptadecanedioic acid, japanic acid, and phellognic acid.

In some embodiments, the linear dicarboxylic acid comprises the reaction product of a saturated linear dicarboxylic acid and a linear polymer. In some embodiments, the saturated linear dicarboxylic acid is one or more selected from the group consisting of: succinic acid, glutaric acid, adipic acid, pimelic acid, suberic acid, azelaic acid, sebacic acid, undecanedioic acid, dodecanedioic acid, brassylic acid, tetradecanedioic acid, pentadecanedioic acid, thapsic acid, heptadecanedioic acid, japanic acid, and phellognic acid. In some embodiments, the saturated linear carboxylic acid is sebacic acid. In some embodiments, the linear polymer is one or more selected from the group consisting of poly(ethylene glycol) (PEG), poly(lactic acid) (PLA), poly(caprolactone) (PCL), poly(glycolic acid) (PGA), poly(lactic-co-glycolic acid) (PLGA), and poly(glycerol sebacate) (PGS). In some embodiments, the linear polymer is PEG. In some embodiments, the PEG is one or more selected from the group consisting of PEG 100, PEG 150, PEG 200, PEG 250, PEG 300, PEG 350, PEG 400, PEG 450, PEG 500, PEG 550, PEG 600, PEG 650, PEG 700, PEG 750, PEG 800, PEG 850, PEG 900, PEG 950, PEG 1000, PEG 1500, PEG 2000, PEG 2500, PEG 3000, PEG 3500, PEG 4000, PEG 4500, and PEG 5000.

In some embodiments, the linear dicarboxylic acid comprises a reaction product that is between about 1% and about 99% saturated linear dicarboxylic acid by weight. In some embodiments, the reaction product is between about 5% and about 90% saturated linear dicarboxylic acid by weight. In some embodiments, the reaction product is between about 10% and about 80% saturated linear dicarboxylic acid by weight. In some embodiments, the reaction product is between about 20% and about 70% saturated linear dicarboxylic acid by weight. In some embodiments, the reaction product is between about 40% and about 60% saturated linear dicarboxylic acid by weight. In some embodiments, the reaction product is about 10%, about 20%, about 30%, about 40%, about 50%, about 60%, about 70%, about 80%, or about 90% saturated linear dicarboxylic acid by weight. In some embodiments, the reaction product is about 50% saturated linear dicarboxylic acid by weight.

In some embodiments, the adhesive composition comprises a reaction product that is between about 1% and about 99% linear dicarboxylic acid by weight. In some embodiments, the reaction product is between about 5% and about 90% linear dicarboxylic acid by weight. In some embodiments, the reaction product is between about 10% and about 90% linear dicarboxylic acid by weight. In some embodiments, the reaction product is between about 30% and about 90% linear dicarboxylic acid by weight. In some embodiments, the reaction product is between about 30% and about 55% linear dicarboxylic acid by weight. In some embodiments, the reaction product is between about 60% and about 90% linear dicarboxylic acid by weight. In some embodiments, the reaction product is about 10%, about 20%, about 30%, about 40%, about 50%, about 60%, about 70%, about 80%, or about 90% linear dicarboxylic acid by weight. In some embodiments, the reaction product is about 50% linear dicarboxylic acid by weight. In some embodiments, the reaction product is about 80% linear dicarboxylic acid by weight.

In some embodiments, one of the reagents used in preparation of the functionalized polymer is a saturated triol. In some embodiments, the saturated triol is selected from the group consisting of: glycerol, 1,2,4-butanetriol, 2-hydroxymethyl-1,3-propanediol, 1,3,5-propanetriol, 2-hydroxymethyl-1,4-butanediol, 1,2,6-hexanetriol, 1,3,6-hexanetriol, 2-hydroxymethyl-1,5-pentanediol, 3-hydroxymethyl-1,5-pentanediol, 1,2,7-heptanetriol, 1,3,7-heptanetriol, 1,4,7-heptanetriol, 2-hydroxymethyl-1,6-hexanediol, 3-hydroxymethyl-1,6-hexanediol, and 3-hydroxyethyl-1,5-pentanediol. In some embodiments, the saturated triol is glycerol.

In some embodiments, the adhesive composition comprises a reaction product that is between about 1% and about 99% saturated triol by weight. In some embodiments, the reaction product is between about 5% and about 80% saturated triol by weight. In some embodiments, the reaction product is between about 10% and about 70% saturated triol by weight. saturated In some embodiments, the reaction product is between about 10% and about 30% saturated triol by weight. In some embodiments, the reaction product is between about 30% and about 40% saturated triol by weight. In some embodiments, the reaction product is about 5%, about 10%, about 15%, about 20%, about 25%, about 30%, about 35%, about 40%, about 45%, about 50%, about 60%, about 70%, about 80%, or about 90% saturated triol by weight. In some embodiments, the reaction product is about 45% saturated triol by weight. In some embodiments, the reaction product comprises about 20% saturated triol by weight.

In some embodiments, one of the reagents used in preparation of the functionalized polymer is an aromatic amino acid. In some embodiments, the aromatic amino acid is of formula (I):

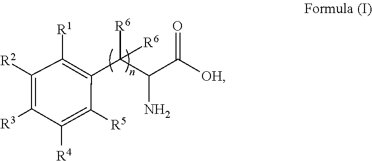

wherein R¹-R⁵ are each independently selected from the group consisting of H, D, F, Cl, Br, hydroxyl, hydroxymethyl, hydroxyethyl, methoxy, ethoxy, methyl, ethyl, amino, aminomethyl, aminoethyl, methylamino, and ethylamino;
wherein at least one of R¹-R⁵ is a hydroxyl;
wherein n=1-6; and
wherein each instance of R⁶ is independently selected from H, D, F, and Cl.

In some embodiments, at least one of R¹-R⁵ is a hydroxyl group. In some embodiments, at least two of R¹-R⁵ are hydroxyl groups. In some embodiments, R¹ and R² are hydroxyl groups. In some embodiments, R² and R³ are hydroxyl groups. In some embodiments, R¹ and R³ are hydroxyl groups. In some embodiments, R¹ and R⁴ are hydroxyl groups. In some embodiments, R¹ and R⁵ are hydroxyl groups. In some embodiments, R² and R⁴ are hydroxyl groups. In some embodiments, at least three of R¹-R⁵ are hydroxyl groups. In some embodiments, R¹, R², and R³ are hydroxyl groups. In some embodiments, R¹, R², and R⁴ are hydroxyl groups. In some embodiments, R¹, R², and R⁵ are hydroxyl groups. In some embodiments, R¹, R³, and R+ are hydroxyl groups. In some embodiments, R¹, R³, and R⁵ are hydroxyl groups. In some embodiments, R², R³, and R⁴ are hydroxyl groups. In some embodiments, at least four of R¹-R⁵ are hydroxyl groups. In some embodiments, R¹, R², R³, and R⁴ are hydroxyl groups. In some embodiments, R¹, R², R³, and R⁵ are hydroxyl groups. In some embodiments, R¹, R², R⁴, and R⁵ are hydroxyl groups. In some embodiments, all of R¹-R⁵ are hydroxyl groups. In some embodiments, R¹, R⁴, and R⁵ are H and R² and R³ are hydroxyl groups.

In some embodiments, each instance of R⁶ is H.

In some embodiments, n=1.

In some embodiments, n=1, R¹, R⁴, and R⁵ are H, R² and R³ are hydroxyl groups, and each instance of R⁶ is H.

In some embodiments, the adhesive composition comprises a reaction product that is between about 0.001 and about 99% aromatic amino acid by weight. In some embodiments, the reaction product is between about 0.01 and about 50% aromatic amino acid by weight. In some embodiments, the reaction product is between about 0.1 and about 25% aromatic amino acid by weight. saturated In some embodiments, the reaction product is between about 1 and about 10% aromatic amino acid by weight. In some embodiments, the reaction product is between about 1 and about 5% aromatic amino acid by weight. In some embodiments, the reaction product is about 0.1, about 0.2, about 0.3, about 0.4, about 0.5, about 0.6, about 0.7, about 0.8, about 0.9, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 11, about 12, about 13, about 14, about 15, about 16, about 17, about 18, about 19, about 20, about 25, about 30, about 35, about 40, about 45, about 50, about 60, about 70, about 80, or about 90% aromatic amino acid by weight. In some embodiments, the reaction product comprises about 3% aromatic acid by weight. In some embodiments, the reaction product comprises about 1% aromatic amino acid by weight.

In some embodiments, the adhesive composition comprises a functionalized polymer. In some embodiments, the functionalized polymer is neutral, cationic, or anionic. In some embodiments, the functionalized polymer is cationic or anionic with respect to its backbone chain. In some embodiments, the polymer is cationic or anionic with respect to its functionalized sidechains.

In some embodiments, the adhesive polymer is characterized by its size. In some embodiments, the polymer has an average molecular weight of between about 300 Da and about 300 kDa. In some embodiments, the polymer has an average molecular weight of between about 1,000 Da and about 100 kDa. In some embodiments, the polymer has an average molecular weight of between about 10 kDa and about 80 kDa. In some embodiments, the polymer has an average molecular weight of between about 30 kDa and about 60 kDa. In some embodiments, the polymer has an average molecular weight of between about 500 Da and about 1,500 Da. In some embodiments, the polymer has an average molecular weight of between about 800 Da and about 1,200 Da. In some embodiments, the polymer has an average molecular weight of about 300 Da, about 350 Da, about 400 Da, about 450 Da, about 500 Da, about 550 Da, about 600 Da, about 650 Da, about 700 Da, about 750 Da, about 800 Da, about 850 Da, about 900 Da, about 950 Da, about 1,000 Da, about 1,100 Da, about 1,200 Da, about 1,300 Da, about 1,400 Da, about 1,500 Da, about 1,600 Da, about 1,700 Da, about 1,800 Da, about 1,900 Da, about 2 kDa, about 2.5 kDa, about 3 kDa, about 3.5 kDa, about 4 kDa, about 4.5 kDa, about 5 kDa, about 6 kDa, about 7 kDa, about 8 kDa, about 9 kDa, about 10 kDa, about 12 kDa, about 14 kDa, about 16 kDa, about 18 kDa, about 20 kDa, about 25 kDa, about 30 kDa, about 35 kDa, about 40 kDa, about 45 kDa, about 50 kDa, about 60 kDa, about 70 kDa, about 80 kDa, about 90 kDa, about 100 kDa, about 120 kDa, about 140 kDa, about 160 kDa, about 180 kDa, about 200 kDa, about 220 kDa, about 240 kDa, about 260 kDa, about 280 kDa, or about 300 kDa.

In some embodiments, the adhesive polymer is a homopolymer. In some embodiments, the polymer is a co-polymer. In some embodiments, the co-polymer is a block co-polymer. In some embodiments, the co-polymer is a random co-polymer. In some embodiments, the co-polymer is an alternate co-polymer. In some embodiments, the co-polymer is a graft co-polymer.

In some embodiments, the adhesive composition is biocompatible. In some embodiments, the adhesive composition is non-toxic. In some embodiments, the adhesive composition reduces cell viability by less than about 50%. In some embodiments, the adhesive composition reduces cell viability by less than about 40%. In some embodiments, the adhesive composition reduces cell viability by less than about 35%. In some embodiments, the adhesive composition reduces cell viability by less than about 30%. In some embodiments, the adhesive composition reduces cell viability by less than about 25%. In some embodiments, the adhesive composition reduces cell viability by less than about 20%. In some embodiments, the adhesive composition reduces cell viability by less than about 15%. In some embodiments, the adhesive composition reduces cell viability by less than about 10%. In some embodiments, the adhesive composition reduces cell viability by less than about 9%. In some embodiments, the adhesive composition reduces cell viability by less than about 8%. In some embodiments, the adhesive composition reduces cell viability by less than about 7%. In some embodiments, the adhesive composition reduces cell viability by less than about 6%. In some embodiments, the adhesive composition reduces cell viability by less than about 5%. In some embodiments, the adhesive composition reduces cell viability by less than about 4%. In some embodiments, the adhesive composition reduces cell viability by less than about 3%. In some embodiments, the adhesive composition reduces cell viability by less than about 2%. In some embodiments, the adhesive composition reduces cell viability by less than about 1%.

In some embodiments, the adhesive composition is biodegradable. In some embodiments, the adhesive composition degrades at least 5% over 30 days. In some embodiments, the adhesive composition degrades at least about 10% over 30 days. In some embodiments, the adhesive composition degrades at least about 11% over 30 days. In some embodiments, the adhesive composition degrades at least about 12% over 30 days. In some embodiments, the adhesive composition degrades at least about 13% over 30 days. In some embodiments, the adhesive composition degrades at least about 14% over 30 days. In some embodiments, the adhesive composition degrades at least about 15% over 30 days. In some embodiments, the adhesive composition degrades less than about 50% over 30 days. In some embodiments, the adhesive composition degrades less than about 40% over 30 days. In some embodiments, the adhesive composition degrades less than about 30% over 30 days. In some embodiments, the adhesive composition degrades less than about 20% over 30 days.

In some embodiments, the adhesive composition does not exhibit excessive swelling when in contact with fluids. In some embodiments, the composition swells less than about 50%. In some embodiments, the composition swells less than about 40%. In some embodiments, the composition swells less than about 30%. In some embodiments, the composition swells less than about 20%. In some embodiments, the composition swells less than about 15%.

In some embodiments, the adhesive composition is characterized by its mechanical properties. In some embodiments, the mechanical properties of the adhesive composition mimic those of a human tissue. In some embodiments, the adhesive composition is characterized by its Young's modulus. In some embodiments, the composition has a Young's modulus of between about 25 and about 1000 kPa. In some embodiments, the composition has a Young's modulus of between about 30 and about 500 kPa. In some embodiments, the composition has a Young's modulus of between about 30 and about 200 kPa. In some embodiments, the composition has a Young's modulus of between about 30 and about 100.

In some embodiments, the adhesive composition is characterized by its tensile fracture stress. In some embodiments, the tensile fracture stress is between about 10 and about 200 kPa. In some embodiments, the tensile fracture stress is between about 10 and about 100 kPa. In some embodiments, the tensile fracture stress is between about 20 and 80 kPa. In some embodiments, the tensile fracture stress is between about 40 and about 70 kPa.

In some embodiments, the adhesive composition is characterized by its tensile strain at failure. In some embodiments, the tensile strain at failure is between about 100 and about 300%. In some embodiments, the tensile strain at failure is between about 150 and 250%. In some embodiments, the tensile strain at failure is between about 200 and about 250%.

In some embodiments, the adhesive composition is characterized by its adhesive strength. In some embodiments, the adhesive strength is determined by lap sheer on collagen sheets. In some embodiments, the adhesive strength is at least about 2 kPa. In some embodiments, the adhesive strength is at least about 3 kPa. In some embodiments, the adhesive strength is between about 2 and about 6 kPa. In some embodiments, the adhesive strength is between about 3 and 5 kPa.

In some embodiments, the adhesive strength is determined by lap sheer on two tissue surfaces. In some embodiments, the adhesive strength is at least about 0.1 kPa. In some embodiments, the adhesive strength is at least about 0.15 kPa. In some embodiments, the adhesive strength is at least about 0.2 kPa. In some embodiments, the adhesive strength is between about 0.1 and about 0.3 kPa. In some embodiments, the adhesive strength is between about 0.15 and about 0.25 kPa.

In some embodiments, the adhesive strength is determined by tensile adhesive testing on two collagen sheets. In some embodiments, the adhesive strength is at least about 1 kPa. In some embodiments, the adhesive strength is at least about 2 kPa. In some embodiments, the adhesive strength is at least about 3 kPa. In some embodiments, the adhesive strength is between about 1 and about 5 kPa. In some embodiments, the adhesive strength is between about 2 and about 4 kPa.

In some embodiments, the adhesive strength is determined by peeling test on two collagen sheets. In some embodiments, the adhesive strength is at least about 20 Pa. In some embodiments, the adhesive strength is at least about 25 Pa. In some embodiments, the adhesive strength is at least about 30 Pa. In some embodiments, the adhesive strength is between about 20 and about 50 Pa. In some embodiments, the adhesive strength is between about 25 and about 40 Pa.

### Methods of Preparation

In some embodiments, the disclosure provides methods of preparing an adhesive composition of the invention. In some embodiments, the method comprises the steps of:
a) combining a linear diacid of interest, a saturated triol of interest, and an aromatic amino acid of interest;
b) stirring the mixture under an inert atmosphere at an elevated temperature; and
c) incubating the mixture under reduced pressure at an elevated temperature.

In some embodiments, in step (a), the linear diacid of interest and saturated triol of interest are utilized in a molar ratio of between about 10:1 to about 1:10. In some embodiments, the ratio is between about 5:1 and about 1:5. In some embodiments, the ratio is between about 2:1 and about 1:3. In some embodiments, the ratio is between about 1:1 and about 1:2. In some embodiments, the ratio is about 10:1, about 9:1, about 8:1, about 7:1, about 6:1, about 5:1, about 4:1, about 3:1, about 2:1, about 1.5:1, about 1:1, about 1:1.5, about 1:2, about 1:3, about 1:4, about 1:5, about 1:6, about 1:7, about 1:8, about 1:9, or about 1:10.

In some embodiments, in step (a), the saturated triol of interest and aromatic amino acid of interest are utilized in a molar ration of between about 1000:1 and about 1:1. In some embodiments, the ratio is between about 500:1 and 2:1. In some embodiments, the ratio is between about 100:1 and about 3:1. In some embodiments, the ratio is about 1000:1, about 950:1, about 900:1, about 850:1, about 800; 1, about 750:1, about 700:1, about 650:1, about 600:1, about 550:1, about 500:1, about 450:1, about 400:1, about 350:1, about 300:1, about 250:1, about 200:1, about 100:1, about 80:1, about 60:1, about 40:1, about 20:1, about 10:1, about 9:1, about 8:1, about 7:1, about 6:1, about 5:1, about 4:1, about 3:1, about 2:1, or about 1:1.

In some embodiments, in step (a), the linear diacid of interest and aromatic amino acid of interest are utilized in a molar ration of between about 500:1 and about 1:1. In some embodiments, the ratio is between about 250:1 and 2:1. In some embodiments, the ratio is between about 200:1 and about 3:1. In some embodiments, the ratio is about 500:1, about 450:1, about 400:1, about 350:1, about 300:1, about 250:1, about 200:1, about 100:1, about 80:1, about 60:1, about 40:1, about 20:1, about 10:1, about 9:1, about 8:1, about 7:1, about 6:1, about 5:1, about 4:1, about 3:1, about 2:1, or about 1:1.

In some embodiments, step (b) occurs at a temperature of at least about 50° C. In some embodiments, step (b) occurs at a temperature of at least about 75° C. In some embodiments, step (b) occurs at a temperature of at least about 100° C. In some embodiments, step (b) occurs at a temperature of at least about 110° C. In some embodiments, step (b) occurs at a temperature of at least about 120° C. In some embodiments, step (b) occurs at a temperature of at least about 130° C. In some embodiments, step (b) occurs at a temperature of about 100° C., about 110° C., about 120° C., about 125° C., about 130° C., about 135° C., or about 140° C.

In some embodiments, the stirring of step (b) is for at least about one hour. In some embodiments, the stirring of step (b) is for at least about two hours. In some embodiments, the stirring of step (b) is for at least about four hours. In some embodiments, the stirring of step (b) is for at least about six hours. In some embodiments, the stirring of step (b) is for at least about eight hours. In some embodiments, the stirring of step (b) is for at least about ten hours. In some embodiments, the stirring of step (b) is for at least about 12 hours. In some embodiments, the stirring of step (b) is for at least about 14 hours. In some embodiments, the stirring of step (b) is for at least about 16 hours. In some embodiments, the stirring of step (b) is for at least about 18 hours. In some embodiments, the stirring of step (b) is for at least about 20 hours. In some embodiments, the stirring of step (b) is for at least about 22 hours. In some embodiments, the stirring of step (b) is for at least about 24 hours. In some embodiments, the stirring of step (b) is for at least about 36 hours. In some embodiments, the stirring of step (b) is for at least about 48 hours. In some embodiments, the stirring of step (b) is for at least about 72 hours. In some embodiments, the stirring of step (b) is for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 48, or about 72 hours.

In some embodiments, step (c) occurs at a temperature of at least about 50° C. In some embodiments, step (c) occurs at a temperature of at least about 75° C. In some embodiments, step (c) occurs at a temperature of at least about 100° C. In some embodiments, step (c) occurs at a temperature of at least about 110° C. In some embodiments, step (c) occurs at a temperature of at least about 120° C. In some embodiments, step (c) occurs at a temperature of at least about 130° C. In some embodiments, step (c) occurs at a temperature of about 100° C., about 110° C., about 120° C., about 125° C., about 130° C., about 135° C., or about 140° C.

In some embodiments, the incubation of step (c) is for at least about one hour. In some embodiments, the incubation of step (c) is for at least about two hours. In some embodiments, the incubation of step (c) is for at least about four hours. In some embodiments, the incubation of step (c) is for at least about six hours. In some embodiments, the incubation of step (c) is for at least about eight hours. In some embodiments, the incubation of step (c) is for at least about ten hours. In some embodiments, the incubation of step (c) is for at least about 12 hours. In some embodiments, the incubation of step (c) is for at least about 14 hours. In some embodiments, the incubation of step (c) is for at least about 16 hours. In some embodiments, the incubation of step (c) is for at least about 18 hours. In some embodiments, the incubation of step (c) is for at least about 20 hours. In some embodiments, the incubation of step (c) is for at least about 22 hours. In some embodiments, the incubation of step (c) is for at least about 24 hours. In some embodiments, the incubation of step (c) is for at least about 36 hours. In some embodiments, the incubation of step (c) is for at least about 48 hours. In some embodiments, the incubation of step (c) is for at least about 72 hours. In some embodiments, the incubation of step (c) is for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 48, about 72 hours, about 96 hours, about 120 hours, or about 144 hours.

In some embodiments, step (c) occurs at a pressure less than about 50 kPa. In some embodiments, step (c) occurs at a pressure less than about 40 kPa. In some embodiments, step (c) occurs at a pressure less than about 30 kPa. In some embodiments, step (c) occurs at a pressure less than about 20 kPa. In some embodiments, step (c) occurs at a pressure less than about 10 kPa. In some embodiments, step (c) occurs at a pressure less than about 9 kPa. In some embodiments, step (c) occurs at a pressure less than about 8 kPa. In some embodiments, step (c) occurs at a pressure less than about 7 kPa. In some embodiments, step (c) occurs at a pressure less than about 6 kPa. In some embodiments, step (c) occurs at a pressure of about 50 kPa, about 40 kPa, about 30 kPa, about 20 kPa, about 10 kPa, about 9 kPa, about 8 kPa, about 7 kPa, about 6 kPa, about 5 kPa, about 4 kPa, about 3 kPa, about 2 kPa, about 1 kPa, about 500 Pa, about 250 Pa, about 200 Pa, about 150 Pa, about 100 Pa, or about 50 Pa.

In some embodiments, step (a) further comprises a step of preparing the linear diacid, comprising the steps of:
a1) combining a saturated linear diacid of interest and a polymer of interest;
b1) stirring the mixture under an inert atmosphere at an elevated temperature; and
c1) incubating the mixture under reduced pressure at an elevated temperature.

In some embodiments, step (b1) occurs at a temperature of at least about 50° C. In some embodiments, step (b1) occurs at a temperature of at least about 75° C. In some embodiments, step (b1) occurs at a temperature of at least about 100° C. In some embodiments, step (b1) occurs at a temperature of at least about 110° C. In some embodiments, step (b1) occurs at a temperature of at least about 120° C. In some embodiments, step (b1) occurs at a temperature of at least about 130° C. In some embodiments, step (b1) occurs at a temperature of about 100° C., about 110° C., about 120° C., about 125° C., about 130° C., about 135° C., or about 140° C.

In some embodiments, the stirring of step (b1) is for at least about one hour. In some embodiments, the stirring of step (b1) is for at least about two hours. In some embodiments, the stirring of step (b1) is for at least about four hours. In some embodiments, the stirring of step (b1) is for at least about six hours. In some embodiments, the stirring of step (b1) is for at least about eight hours. In some embodiments, the stirring of step (b1) is for at least about ten hours. In some embodiments, the stirring of step (b1) is for at least about 12 hours. In some embodiments, the stirring of step (b1) is for at least about 14 hours. In some embodiments, the stirring of step (b1) is for at least about 16 hours. In some embodiments, the stirring of step (b1) is for at least about 18 hours. In some embodiments, the stirring of step (b1) is for at least about 20 hours. In some embodiments, the stirring of step (b1) is for at least about 22 hours. In some embodiments, the stirring of step (b1) is for at least about 24 hours. In some embodiments, the stirring of step (b1) is for at least about 36 hours. In some embodiments, the stirring of step (b1) is for at least about 48 hours. In some embodiments, the stirring of step (b1) is for at least about 72 hours. In some embodiments, the stirring of step (b1) is for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 48, or about 72 hours.

In some embodiments, step (c1) occurs at a temperature of at least about 50° C. In some embodiments, step (c1) occurs at a temperature of at least about 75° C. In some embodiments, step (c1) occurs at a temperature of at least about 100° C. In some embodiments, step (c1) occurs at a temperature of at least about 110° C. In some embodiments, step (c1) occurs at a temperature of at least about 120° C. In some embodiments, step (c1) occurs at a temperature of at least about 130° C. In some embodiments, step (c1) occurs at a temperature of about 100° C., about 110° C., about 120° C., about 125° C., about 130° C., about 135° C., or about 140° C.

In some embodiments, the incubation of step (c1) is for at least about 10 minutes. In some embodiments, the incubation of step (c1) is for at least about 20 minutes. In some embodiments, the incubation of step (c1) is for at least about 30 minutes. In some embodiments, the incubation of step (c1) is for at least about 40 minutes. In some embodiments, the incubation of step (c1) is for at least about 50 minutes. In some embodiments, the incubation of step (c1) is for at least about 60 minutes. In some embodiments, the incubation of step (c1) is for at least about 90 minutes. In some embodiments, the incubation of step (c1) is for at least about 120 minutes. In some embodiments, the incubation of step (c1) is about 10, about 20, about 30, about 40, about 50, about 60, about 70, about 80, about 90, about 100, about 110, or about 120 minutes.

In some embodiments, step (c1) occurs at a pressure less than about 50 kPa. In some embodiments, step (c1) occurs at a pressure less than about 40 kPa. In some embodiments, step (c1) occurs at a pressure less than about 30 kPa. In some embodiments, step (c1) occurs at a pressure less than about 20 kPa. In some embodiments, step (c1) occurs at a pressure less than about 10 kPa. In some embodiments, step (c1) occurs at a pressure less than about 9 kPa. In some embodiments, step (c1) occurs at a pressure less than about 8 kPa. In some embodiments, step (c1) occurs at a pressure less than about 7 kPa. In some embodiments, step (c1) occurs at a pressure less than about 6 kPa. In some embodiments, step (c1) occurs at a pressure of about 50 kPa, about 40 kPa, about 30 kPa, about 20 kPa, about 10 kPa, about 9 kPa, about 8 kPa, about 7 kPa, about 6 kPa, about 5 kPa, about 4 kPa, about 3 kPa, about 2 kPa, about 1 kPa, about 500 Pa, about 250 Pa, about 200 Pa, about 150 Pa, about 100 Pa, or about 50 Pa.

### Sealants

The present invention, in part, relates to sealants, or wound closure materials. In some embodiments, a sealant comprises an adhesive composition of the present invention. In some embodiments, a sealant comprises a single layer of an adhesive composition of the present invention.

A sealant of the present invention may be of any shape or dimensions appropriate for its application. Appropriate shapes include, but are not limited to, circles, ovals, squares, rectangles, “dog bones”, “butterflies”, “hourglasses”, “H-shapes”, “X-shapes”, etc. As an example, a sealant may be a circular pad with a radius between about 1 mm and about 10 cm and a thickness between about 10 μm and about 10 mm.

In some embodiments, a sealant comprises two or more layers of an adhesive composition of the present invention. In some embodiments, the two or more layers comprise the same adhesive composition. In some embodiments, the two or more layers comprise at least two different adhesive compositions of the present invention. In some embodiments, the two or more different adhesive compositions have been formulated to increase or decrease the adhesive strength of certain areas of the sealant. For example, the first layer of a two-layer sealant may be formulated to have an adhesive strength of about 0.5 kPa and the second layer may be formulated to have an adhesive strength of about 4 kPa. In some embodiments, the two or more different adhesive compositions have been formulated to have different mechanical properties. As an example, the second layer of a two-layer sealant may be formulated to be more elastic than the first layer.

In some embodiments, the two or more layers of adhesive compositions are of the same shape. In some embodiments, the two or more layers of adhesive compositions are of the same thickness. In some embodiments, the two or more layers are of different shapes and/or thicknesses. As an example, a first layer of sealant may be a circular pad with a radius of 5 mm and a thickness of 1 mm, centered in a second layer that is a circular pad with a radius of 2 cm and a thickness of 1.5 mm.

### Adhesive Layers for Closure Devices

The present invention relates, in part, to a self-adhesive devices utilizing the compositions presented herein. In some embodiments, the self-adhesive device comprises a base layer and an adhesive layer. In some embodiments, the adhesive layer of the device comprises an adhesive composition of the invention.

In some embodiments, the base layer is made of a stretchable, porous, and/or breathable material. In some embodiments, the base layer is made of any suitable material. In some embodiments, the base is made of a material that is transparent, or substantially transparent, thus allowing for non-invasive monitoring of wound healing. In other embodiments, the base is made of a material that is not transparent. In some embodiments, the base is made from natural, synthetic, and/or artificial materials. In some embodiments, the base is comprised of materials that are nontoxic, biodegradable, bioresorbable, and/or biocompatible. In some embodiments, the base comprises inert materials. In some embodiments, the base comprises activated materials, (e.g., activated carbon cloth to remove microbes, as disclosed in WO2013028966A2, incorporated herein by reference in its entirety).

In some embodiments, the base comprises one or more materials selected from the group consisting of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, hydrogel, catechol-modified hydrogel, Gore-Tex, plastic and plastic components, polymers, biopolymers, metals (e.g., gold, silver, platinum, steel or other alloys); metal-coated materials; metal oxides; plastics; ceramics; silicon; glasses; mica; graphite and natural materials.

In some embodiments, the base layer comprises a polymer. In some embodiments, the polymer is biocompatible. In some embodiments, the polymer is biodegradable. In some embodiments, the polymer is a neutral polymer, cationic polymer, anionic polymer, or any combination thereof. In some embodiments, the polymer is a homopolymer or co-polymer. In some embodiments, the polymer comprises one or more selected from the group consisting of polylactic acid (PLA), poly(L-lactic acid) (PLLA), poly(D,L-lactic acid) (PDLLA), polycaprolactone (PCL), chitosan, polyglycolic acid (PGA), poly(lactic-go-glycolic acid) (PLGA), poly(3-hydroxybuterate-co-3-hydroxyvalerate (PHBV), chitin, polyhydroxyalkanoates (PHAs), polyamides, poly(glycerol sebacate) (PGS), polydioxanone (PDS), poly(trimethylene carbonate) (PTMC), cyanophycin, PEG, polyethylene glycol diacrylate (PEGDA), poly(vinyl alcohol) (PVA), poly(lactic acid-co-caprlocactone) (PLCL), poly(ester urea) (PEU), poly(ester urethane), and combinations thereof. In some embodiments, the polymer is PGS. In some embodiments, the polymer is modified. In some embodiments, the polymer is modified to comprise catechol groups.

In some embodiments, the device can comprise a magnetic material. For example, a magnetic material can be utilized for positioning the device in a target site or orientation, to trigger delivery of a therapeutic agent, or to affect interaction of the adhesive layer to an internal tissue or a vessel wall.

In some embodiments, the device comprises deformable materials (e.g., polymers). As an example, the device can comprise a deformable rubber so that the device swells, enabling interaction of the adhesive layer protruding from the base layer to a tissue. In another example, a deformable device may be able to change size depending on pressure so that it can pass through lumens with diameters smaller than that of the device.

In some embodiments, the base layer is flexible. The degree of flexibility of the base layer is determined by the material of construction, the shape and dimensions of the device, the type and properties of the approximated tissue, and the area of the body into which device is placed. For example, a tightly curved or mobile part of the body, e.g. a joint, may require a more flexible base, as would a tendon or nerve repair due to the amount of bending the device needs for the attachment. Also, depending on the type of material used, the thickness of the base, as well as its width and length, may determine the flexibility of the device. The length and width of the base can be in a range between about 1 mm to 10 cm. Thickness of the base can be in a range between about 10 μm to 1 cm. The base may be pre-fabricated into different shapes. In one embodiment, the base has sharp corners. In one embodiment, the base has round corners. The shape and dimensions of the base can be modified to change the flexibility of the device.

In some embodiments, the adhesive layer fully or partially covers the interacting surface of the base layer. In some embodiments, the adhesive layer fully covers the interacting surface of the base layer. In some embodiments, the adhesive layer covers between about 10 and about 90 percent of the interacting surface of the base layer. In some embodiments, the adhesive layer covers between about 20 and about 80 percent of the interacting surface of the base layer. In some embodiments, the adhesive layer covers between about 30 and about 70 percent of the interacting surface of the base layer. In some embodiments, the adhesive layer covers between about 40 and about 60 percent of the interacting surface of the base layer. In some embodiments, the adhesive layer covers about 10, about 20, about 30, about 40, about 50, about 60, about 70, about 80, about 90, about 95, about 96, about 97, about 98, about 99, about 99.5, or about 99.9 percent of the interacting surface of the base layer.

In some embodiments, the self-adhesive device is biocompatible. In some embodiments, the self-adhesive device is biodegradable. In some embodiments, the self-adhesive device is non-toxic.

In some embodiments, the self-adhesive device is characterized by its mechanical properties. In some embodiments, the mechanical properties of the device mimic those of a human tissue. In some embodiments, the device is characterized by its Young's modulus. In some embodiments, the device has a Young's modulus of between about 30 and about 1000 kPa. In some embodiments, the device has a Young's modulus of between about 30 and about 500 kPa. In some embodiments, the device has a Young's modulus of between about 30 and about 200 kPa. In some embodiments, the device has a Young's modulus of between about 30 and about 100 kPa.

In some embodiments, the self-adhesive device is characterized by its tensile fracture stress. In some embodiments, the tensile fracture stress is between about 10 and about 200 kPa. In some embodiments, the tensile fracture stress is between about 10 and about 100 kPa. In some embodiments, the tensile fracture stress is between about 20 and 80 kPa. In some embodiments, the tensile fracture stress is between about 40 and about 70 kPa.

In some embodiments, the self-adhesive device is characterized by its tensile strain at failure. In some embodiments, the tensile strain at failure is between about 100 and about 500%. In some embodiments, the tensile strain at failure is between about 150 and 300%. In some embodiments, the tensile strain at failure is between about 200 and about 250%.

In some embodiments, the self-adhesive device is characterized by the adhesive strength of its adhesive layer. In some embodiments, the adhesive strength is determined by lap sheer on collagen sheets. In some embodiments, the adhesive strength is at least about 2 kPa. In some embodiments, the adhesive strength is at least about 3 kPa. In some embodiments, the adhesive strength is between about 2 and about 6 kPa. In some embodiments, the adhesive strength is between about 3 and 5 kPa.

In some embodiments, the adhesive strength is determined by lap sheer on two tissue surfaces. In some embodiments, the adhesive strength is at least about 0.1 kPa. In some embodiments, the adhesive strength is at least about 0.15 kPa. In some embodiments, the adhesive strength is at least about 0.2 kPa. In some embodiments, the adhesive strength is between about 0.1 and about 0.3 kPa. In some embodiments, the adhesive strength is between about 0.15 and about 0.25 kPa.

In some embodiments, the adhesive strength is determined by tensile adhesive testing on two collagen sheets. In some embodiments, the adhesive strength is at least about 1 kPa. In some embodiments, the adhesive strength is at least about 2 kPa. In some embodiments, the adhesive strength is at least about 3 kPa. In some embodiments, the adhesive strength is between about 1 and about 10 kPa. In some embodiments, the adhesive strength is between about 2 and about 4 kPa.

In some embodiments, the adhesive strength is determined by peeling test on two collagen sheets. In some embodiments, the adhesive strength is at least about 20 Pa. In some embodiments, the adhesive strength is at least about 25 Pa. In some embodiments, the adhesive strength is at least about 30 Pa. In some embodiments, the adhesive strength is between about 20 and about 50 Pa. In some embodiments, the adhesive strength is between about 25 and about 40 Pa.

In some embodiments, the self-adhesive device is characterized by its burst pressure. In some embodiments, the burst pressure is at least about 5 kPa. In some embodiments, the burst pressure is at least about 10 kPa. In some embodiments, the burst pressure is at least about 15 kPa. In some embodiments, the burst pressure is at least about 16 kPa. In some embodiments, the burst pressure is at least about 17 kPa. In some embodiments, the burst pressure is at least about 18 kPa. In some embodiments, the burst pressure is at least about 19 kPa.

In some embodiments, the self-adhesive device is a patch. In some embodiments, the self-adhesive device is a bandage. In some embodiments, the self-adhesive device is a surgical closure device. In some embodiments, the self-adhesive device is a sustained drug release device.

In some embodiments, a self-adhesive device is a bio-zipper surgical closure device, an implantable wound closure device for use in a subject. In one embodiment, the bio-zipper surgical closure device provides tension-free support of an incision throughout the healing process. In one embodiment, the present invention provides a bio-zipper surgical closure device suitable for urethral tubular closure during a urethroplasty. The bio-zipper device is designed to facilitate epithelial inversion, minimize urine leak, alleviate tension along the full extent of a ventral urethral closure site, and prevent localized laminar flow effects.

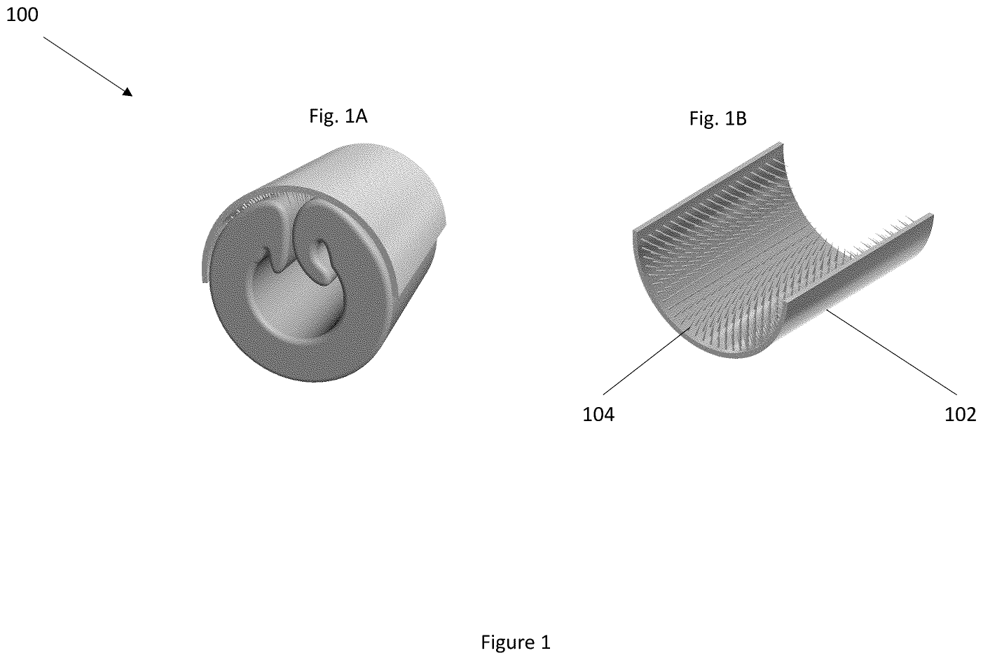

Referring to FIG. 1A and FIG. 1B, an exemplary bio-zipper device 100 of the present invention is shown. Bio-zipper 100 comprises a base 102 and an adhesive layer 104 on and/or protruding from base 102. In various embodiments, the adhesive layer comprises at least one adhesive polymer and a plurality of microstructures, or a combination thereof.

Alternatively, the flexibility and/or stretchability of base 102 may vary across, or along, bio-zipper 100. Further, in some embodiments, base 102 can comprise elastic properties, wherein the elasticity may optionally be similar throughout base 102. Alternatively, the elasticity may be varied along or across base 102.

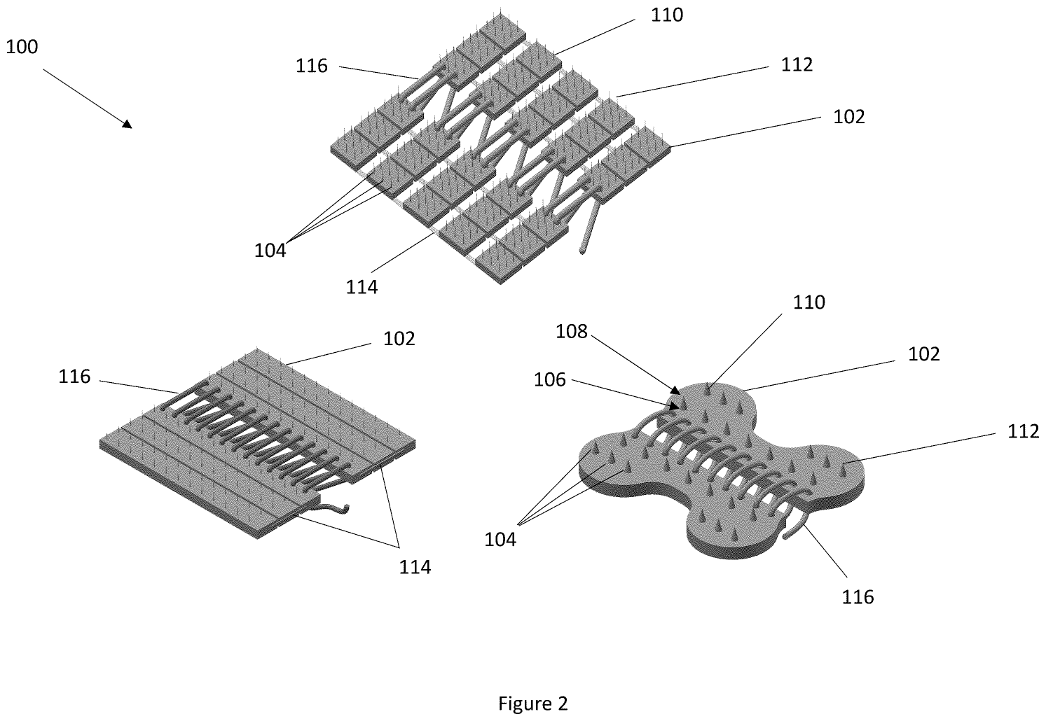

Referring now to FIG. 2, shown are exemplary biozipper 100 according to aspects of the present invention. In some embodiments, adhesive layer 104 each comprise a proximal end 106, a distal end 108, a body 110 and a tip 112. Adhesive layer 104 can be either straight or curved. In some embodiments, body 110 connects proximal end 106 to distal end 108 without curvature along its length. In one embodiment, body 110 is curved along its length between proximal end 106 to distal end 108.

Microstructures of adhesive layer 104 may be canted or erect. In one embodiment, the general structure of microstructures of adhesive layer 104 is of a rose thorn shape. In one embodiment, microstructures of adhesive layer 104 are selected from the group consisting of microneedles, microblades, microanchors, microfishscale, micropillars, microhairs, and any combination thereof. Microstructures of adhesive layer 104 can have a sharp tip 112 enabling it to penetrate into tissue, or can have a blunt tip 112 that enables it to merely grasp tissue without actual penetration. In one embodiment, microstructures of adhesive layer 104 are designed to penetrate tissue to specific depths.

Microstructures of adhesive layer 104 can have a circular cross-section or non-circular cross-section at proximal end 106. The cross-sectional dimensions typically are between about 10 nm and 1 mm, preferably between about 1 micron and 200 microns, and more preferably between about 10 and 100 μm.

The bio-zipper 100 of the present invention may comprise adhesive layer 104 of any desired size, dimension, and geometry. Additionally, adhesive layer 104 may optionally comprise surfaces which are substantially smooth, or which comprise uneven surfaces, e.g., a microstructure comprising sides which are wavy, or which comprise protrusions, indentations, or depressions. For example, body 110 can have concave surfaces, convex surfaces, or a combination of concave and convex surfaces. In one embodiment, body 110 comprises at least one concave surface. In one embodiment, body 110 comprises at least one convex surface. In one embodiment, body 110 comprises at least one concave surface and at least one convex surface.

Tip 112 is located at distal end 108. In one embodiment, tip 112 can be selected from a group consisting of: a cube, a rectangle, a sphere, a cone, a pyramid, a cylinder, a tube, a ring, a tetrahedron, a hexagon, an octagon, or any irregular shapes. In one embodiment, the dimension (e.g., a diameter) of tip 112 may be within a range of about 10 nm to 1 μm.

The density, distribution, length, and orientation of microstructures of adhesive layer 104 on base 102 may be modified depending on the type of wound closure. Microstructures of adhesive layer 104 may be bent or curve gradually, with distal end 108 directed at an optimal angle relative to base 102 to aid device penetration and stability within the tissue, and to reduce tissue irritation after installation. Microstructures of adhesive layer 104 may be canted in one direction, such as toward the center of bio-zipper 100. Microstructures of adhesive layer 104 may also be variously oriented, such as toward center and erect, or toward center and away from center. It is within the scope of this invention to have microstructures of adhesive layer 104 extending in any relative direction or orientation on base 102.

In one embodiment, bio-zipper 100 comprises microstructures of adhesive layer 104 at an angle relative to base 102. Microstructures of adhesive layer 104 may be positioned at any suitable angle. In one embodiment, microstructures of adhesive layer 104 are affixed at an angle relative to base 102, wherein the angle is approximately 15, 30, 45, 60, 75, or 90 degrees, including all integers (e.g., 16°, 17°, 18°, etc.) and ranges (e.g., 15°-90°, 30°-90°, 45°-70°, etc.) in between of the angles set forth. In one embodiment, bio-zipper 100 of the present invention also include microstructures of adhesive layer 104 with an angle relative to base 102, that is variable depending on its position in any microstructure array. In certain embodiments, the angle of one or more microstructures of adhesive layer 104 is approximately constant along the entire length of the microstructure of adhesive layer 104, and in other embodiments, the angle of the microstructure of adhesive layer 104 varies along the length of the microstructure of adhesive layer 104.

Microstructures of adhesive layer 104 may be angled in any direction. In some embodiments, all microstructures of adhesive layer 104 in a particular array are angled in the same direction, or in approximately the same direction; while in other embodiments they are not.

In one embodiment, microstructures of adhesive layer 104 of various lengths emanate from a single base 102. For example, in one embodiment, microstructures of adhesive layer 104 are progressively shorter the closer they are to the center of bio-zipper 100. In one embodiment, microstructures of adhesive layer 104 may also become progressively shorter the farther they are from the center of bio-zipper 100. In one embodiments, the length of an individual microstructure of adhesive layer 104 may be within a range of about 1 μm to 2 mm. It may be desirable, in certain embodiments, to adjust the length of a microneedle according to the application/use and/or a payload delivered by bio-zipper 100.

The density of microstructures of adhesive layer 104 may be predetermined and may vary depending upon the size of bio-zipper 100 and the wound to be closed, much as bandages vary in size and the location on the body where they are to be applied. In one embodiment, the density may be about or greater than about 100,000/cm², about 10,000/cm², about 5,000/cm², about 1,000/cm², about 500/cm², about 100/cm², about 50/cm², about 10/cm², or even about 1/cm². The pitch between adjacent microneedles may be from about 10 μm to more than 1 cm, wherein pitch is defined as the distance between microstructures of adhesive layer 104, center point to center point.

Without wishing to be bound by any particular theory, the degradable portion of adhesive layer 104 and the degradation rate may dictate the mechanism and efficiency of delivery of a therapeutic agent or other functions of bio-zipper 100. For instance, adhesive layer 104 can include or introduce a therapeutic agent so that the therapeutic agent is released after the degradation of adhesive layer 104. In one embodiment, base 102 comprises a degradable material. In certain embodiments, base 102 degrades so that adhesive layer 104 is released from bio-zipper 100 and may remain lodged in the internal tissue after interaction and/or implantation. In certain embodiments, adhesive layer 104 lodged in the internal tissue may gradually degrade. In one embodiment, tip 112 comprises a degradable material. In one embodiment, tip 112 of an adhesive layer 104 degrades so that only tip 112 of the adhesive layer 104 breaks off. In one embodiment, adhesive layer 104 may be coated with a therapeutic agent. In one embodiment, base 102 may be coated with a therapeutic agent.

Bio-zipper 100 may be molded, stamped, machined, woven, bent, welded, cut, formed, or otherwise fabricated to create the desired features and functional properties.

Referring again to FIG. 2, another exemplary bio-zipper surgical wound closure device is shown.

In one embodiment, a plurality of bio-zippers 100 can be attached together via a flexible backbone 114. Backbone 114 can attach to the plurality of bio-zippers 100 by any means, including but not limited to adhesives, snap fits, etc. Backbone 114 can be made of any suitable material. In some embodiments, for example, Backbone 114 can be made from natural, synthetic, and/or artificial materials; and in some particular embodiments, backbone 114 can comprise a polymeric substance (e.g., a silicone, a polyurethane, or a polyethylene). Backbone 114 may comprise materials that are nontoxic, biodegradable, bioresorbable, or biocompatible.

For example, in one embodiment, backbone 114 comprises a polymer. In one embodiment, the polymer is a biodegradable polymer. In some embodiments, the polymer is a neutral polymer, cationic polymer, anionic polymer, or any combination thereof. In some embodiments, the polymer is a homopolymer or co-polymer. Examples of such polymers include, but are not limited to, polyethylene glycol (PEG), functionalized PEG, polyglycerol, functionalized polyglycerol, poly(glycerol sebacate) (PGS), functionalized PGS, L-3,4-dihydroxyphenylalanine (L-DOPA) terminated polymer, L-DOPA terminated co-polymer, sebacic acid terminated polymer, sebacic acid terminated co-polymer, or any combination thereof. For example, in one embodiment, backbone 114 comprises poly(glycerol sebacate) (PGS).

In one embodiment, a plurality of bio-zippers 100 are placed adjacent together leaving a space between each bio-zipper 100 ranging between about 0 to 1 cm. This space allows the bio-zippers 100 to move flexibly and bend based on the location of the application site.

In one embodiment, at least two bio-zippers 100 can be attached together via a closure member 116. Closure member 116 allows the at least two bio-zippers 100 to be drawn closer together using sutures, threads, pull tabs, or any other mechanism known to the skilled artisan. The action of drawing the plurality of bio-zipper together causes the edges of the tissue opening to be brought toward each other and allows certain embodiments to be effectively applied to tissue openings of varying sizes.

In some embodiments, a self-adhesive device is a biotape surgical closure device, an implantable wound closure device for use in a subject. In one embodiment, the biotape surgical closure device provides tension-free support of an incision throughout the healing process. In one embodiment, the present invention provides a biotape surgical closure device suitable for urethral tubular closure during a urethroplasty. In one embodiment, the biotape device is designed to facilitate epithelial inversion, minimize urine leak, alleviate tension along the full extent of a ventral urethral closure site, and prevent localized laminar flow effects. In one embodiment, the biotape surgical closure device provides a water-tight surgical closure. In one embodiment, the biotape surgical closure device provides one or more channels for drainage of fluids. In one embodiment, the biotape surgical closure device provides one or more viewing windows.

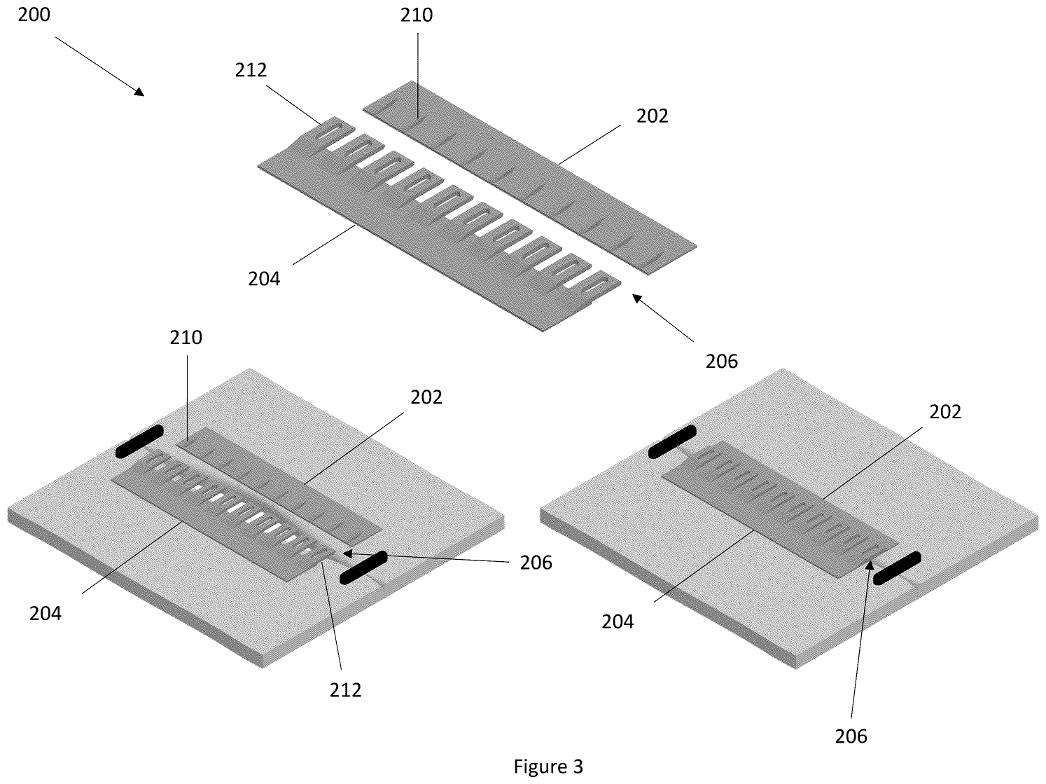

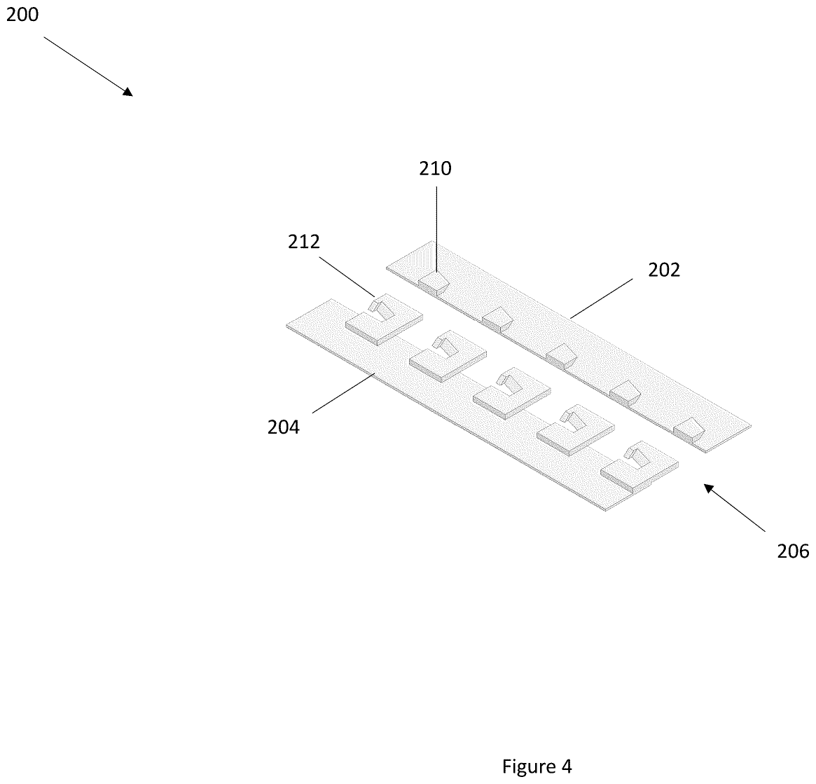

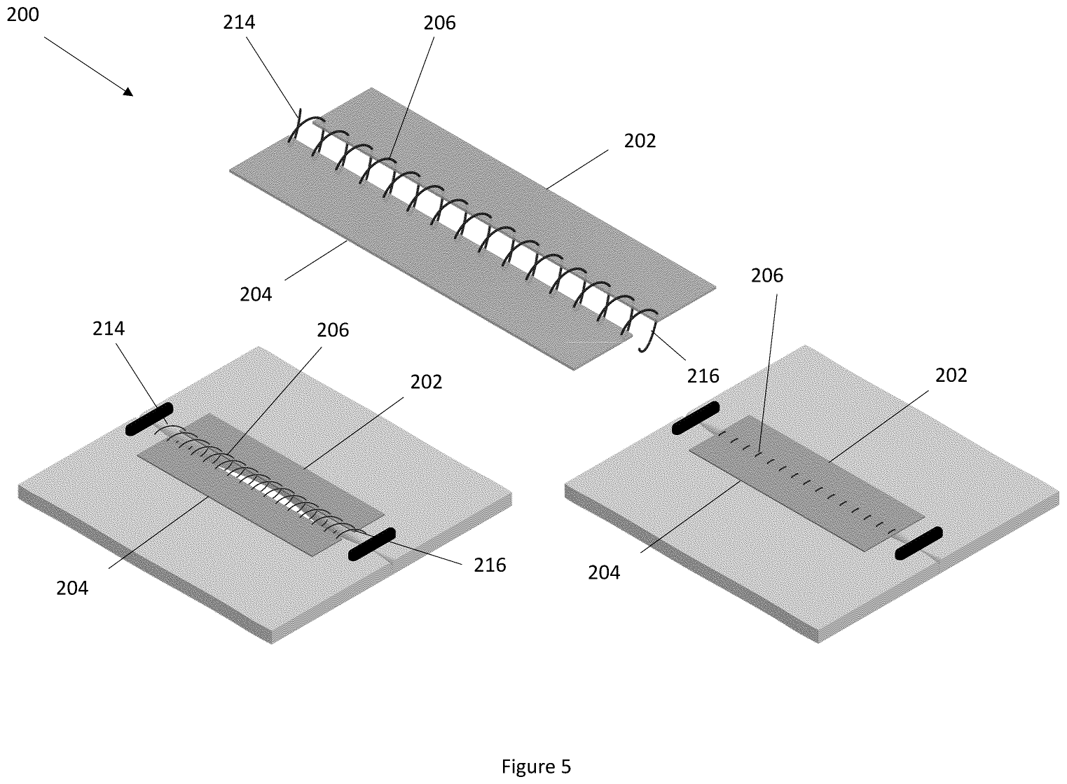

Referring now to FIG. 3, FIG. 4 and FIG. 5, an exemplary biotape device 200 of the present invention is shown. Biotape 200 comprises a right panel 202, a left panel 204 and a closure member 206.

Right panel 202 comprises a lower surface and an upper surface. Similarly, left panel 204 comprises a lower surface and an upper surface.

Right panel 202 and left panel 204 can be made of a stretchable and breathable material. Alternatively, right panel 202 and left panel 204 can be made of any suitable material. In some embodiments, for example, right panel 202 and left panel 204 can be made of a material that is transparent, or substantially transparent, thus allowing for non-invasive monitoring of wound healing. In other embodiments, right panel 202 and left panel 204 can be made of a material that is not transparent. In one embodiment, right panel 202 and left panel 204 may be made from natural, synthetic, and/or artificial materials; and in some particular embodiments, they comprise a polymeric substance (e.g., a silicone, a polyurethane, or a polyethylene).

Right panel 202 and left panel 204 may comprise any material or mixture of materials. In one embodiment, Right panel 202 and left panel 204 can comprise one or more biocompatible materials.

In some embodiments, right panel 202 and left panel 204 can comprise deformable materials (e.g., polymers). As an example, right panel 202 and left panel 204 can comprise a deformable rubber so that a volume of biotape 200 can respond to external pressure. In another example, a deformable right panel 202 and left panel 204 may be able to change size depending on pressure so that it can pass through lumens with diameters smaller than that of the device.

In one embodiment, right panel 202 and left panel 204 may comprise an adhesive surface on their respective lower surfaces. In one embodiment, bottom surfaces of right panel 202 and left panel 204 may be covered with a pressure-responsive adhesive, where the adhesive is initially covered with a protective layer which may be peeled away immediately prior to use. In one embodiment, biotape 200 may further comprise pull-away tabs or other similar structures to hold right panel 202 and left panel 204 together at a pre-determined spaced apart distance after the protective layer has been removed but prior to adhering the panels to tissue surface.

In one embodiment, right panel 202 and left panel 204 may be made from a material with adhesive properties. In one embodiment, right panel 202 and left panel 204 having adhesive properties minimizes risk of delamination and improves mechanical stability of the device when in place.

Closure member 206 allows right panel 202 and left panel 204 to be drawn closer together using sutures, pull tabs, or any other mechanism known to the skilled artisan. The action of drawing right panel 202 and left panel 204 together causes the edges of the tissue opening to be brought toward each other and allows certain embodiments to be effectively applied to tissue openings of varying sizes.

Referring now to FIG. 3 and FIG. 4, an exemplary biotape surgical closure device 200 of the present invention is shown. In one embodiment, closure member 206 may comprise a right member 210 and a left member 212. Right member 210 is secured to an upper surface of right panel 202 and left member 212 is secured to an upper surface of left panel 204. In one embodiment, right member 210 and left member 212 are configured to couple together through a variety of coupling interfaces and bring the edges of the tissue opening toward each other. In one embodiment, the coupling interface is a snap fit mechanism (FIG. 3 and FIG. 4). Other locking interfaces, mechanisms or structures may include but are not limited to resealable adhesive layers, slide locks, locking pins and the like.

Referring now to FIG. 5, another exemplary biotape surgical closure device 200 is shown. In one embodiment, closure member 206 may comprise a continuous strap attached to the edges of right panel 202 and left panel 204 along each panel's length, configured to bring the edges of the tissue opening toward each other. In one embodiment, a continuous strap comprises a first end 214 and a second end 216. The strap may be placed into tension by pulling first end 214 and second end 216, such that the tensioned strap exerts a laterally compressive force on right panel 202 and left panel 204 and thereby the tissue panels they are applied on. The laterally compressive force may promote healing while inhibiting scar formation. In one embodiment, first end 214 and second end 216 may be secured by any means known to one skilled in the art including but not limited to ties. In one embodiment, straps may include but are not limited to nylon or polypropylene line, suture material, string, a cable, a wire, or other similar items.

In one embodiment, closure member 206 may comprise a series of lateral ties attached to the edges of right panel 202 and left panel 204, configured to bring the edges of the tissue opening toward each other. In one embodiment, closure member 206 may comprise a plurality of independent lateral ties fixed to one panel and being adjustably attachable to the other panel. In one exemplary embodiment, the adjustably attachable end may comprise a ratchet tightening mechanism or similar structure which allows each lateral tie to be independently adjusted at a different spacing between right and left panels 202 and 204. In this way, right and left panels 202 and 204 may be differentially tensioned along their inner edges in order to control and optimize the forces applied to the adjacent tissue edges which are being drawn together. In one embodiment, lateral ties may include but are not limited to nylon or polypropylene line, suture material, string, a cable, a wire, or other similar items.

Biotape 200 may be molded, stamped, machined, woven, bent, welded, cut, formed or otherwise fabricated to create the desired features and functional properties.

### Therapeutic Agents

A therapeutic agent can be in a gas form, a liquid form, a solid form, or any combination thereof. In some embodiments, the volume of a therapeutic agent may be in a range of about 0.1 mL to about 50 mL. In certain embodiments, a therapeutic agent of the disclosed self-adhesive device is carried in or transported through adhesive layer 104. An exemplary volume of a therapeutic agent carried within adhesive layer 104 can be within a range of about 1 nL to about 1 μL.

In accordance with the present disclosure, a therapeutic agent can include one or more agents for delivery after administration/implantation. A wide range of agents may be used. Agents may include, but are not limited to, therapeutic agents and/or an imaging agent. For example, agents may comprise any therapeutic agents (e.g. antibiotics, NSAIDs, angiogenesis inhibitors, neuroprotective agents, chemotherapeutic agents), cytotoxic agents, diagnostic agents (e.g. sensing agents, contrast agents; radionuclides; and fluorescent, luminescent, and magnetic moieties), prophylactic agents (e.g. vaccines), and/or nutraceutical agents (e.g. vitamins, minerals, etc.), or other substances that may be suitable for introduction to biological tissues, including pharmaceutical excipients and substances for cosmetics, and the like. In some embodiments, a therapeutic agent includes one or more bioactive agents.

An agent may comprise small molecules, large (i.e., macro-) molecules, or a combination thereof. Additionally or alternatively, an agent can be a formulation including various forms, such as liquids, liquid solutions, gels, hydrogels, solid particles (e.g., microparticles, nanoparticles), or any combination thereof.

In representative non-limiting embodiments, an agent can be selected from among amino acids, vaccines, antiviral agents, nucleic acids (e.g., siRNA, RNAi, and microRNA agents), gene delivery vectors, interleukin inhibitors, immunomodulators, neurotropic factors, neuroprotective agents, antineoplastic agents, chemotherapeutic agents, polysaccharides, anti-coagulants, antibiotics, analgesic agents, anesthetics, antihistamines, anti-inflammatory agents, vitamins and/or any combination thereof. In some embodiments, an agent may be selected from suitable proteins, peptides and fragments thereof, which can be naturally occurring, synthesized or recombinantly produced.

In some embodiments, an agent can comprise a cell. Such a device can be useful for the injection of whole cells (e.g., stem cells). In some embodiments, an agent comprises a biologic. Examples of biologics including, but are not limited to, monoclonal antibodies, single chain antibodies, aptamers, enzymes, growth factors, hormones, fusion proteins, cytokines, therapeutic enzymes, recombinant vaccines, blood factors, and anticoagulants. Exemplary biologics suitable for use in accordance with the present disclosure are discussed in S. Aggarwal, Nature Biotechnology, 28:11, 2010, the contents of which are incorporated by reference herein.

A therapeutic agent used in accordance with the present application can comprise an agent useful in combating inflammation and/or infection. A therapeutic agent may be an antibiotic. Exemplary antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof. Other anti-microbial agents such as copper may also be used in accordance with the present invention. For example, anti-viral agents, anti-protazoal agents, anti-parasitic agents, etc. may be of use. Additionally or alternatively, a therapeutic agent may be an anti-inflammatory agent.

A therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. In another example, an antibiotic may be combined with an inhibitor of the enzyme commonly produced by bacteria to inactivate the antibiotic (e.g., penicillin and clavulanic acid).

In some embodiments, a therapeutic agent may be any therapeutic gene as known in the art. In some embodiments, a therapeutic agent is a non-viral vector. Typical non-viral gene delivery vectors comprise DNA (e.g., plasmid DNA produced in bacteria) or RNA. In certain embodiments, non-viral vectors are used in accordance with the present invention with the aid of a delivery vehicle. Delivery vehicles may be based around lipids (e.g., liposomes) which fuse with cell membranes releasing a nucleic acid into the cytoplasm of the cell. Additionally or alternatively, peptides or polymers may be used to form complexes (e.g., in form of particles) with a nucleic acid which may condense as well as protect the therapeutic activity as it attempts to reach a target destination.

Alternatively, a therapeutic agent can include one or more surfactants. Various surfactants are known in the art and can be suitable for use as an enhancer to increase tissue permeability for delivery.

A therapeutic agent used in accordance with the present application can comprise an agent useful in promoting cell migration and proliferation.

### Coatings

In accordance with the present disclosure, a self-adhesive device comprises a coating. In some embodiments, the surface of the device is coated. In some embodiments, a portion of the device is coated. In some embodiments, a portion of bio-zipper 100 is coated, such as one or more adhesive layers 104. In some embodiment, a portion of biotape 200, such as at least one of right panel 202 and left panel 204 is coated. In some embodiments, base 102 is coated. It will be appreciated that a coating may comprise one or more materials/units/layers.

In some embodiments, a coating comprises a payload, which may include one or more agents for delivery. A coating may be a medicated coating being made of or including an agent such as an anti-microbial agent. For example, an anti-microbial agent (e.g., gentamicin, clindamycin, copper, copper ions, silver) and/or a material with an ability to induce anti-microbial activity (e.g., gold that can be heated with an electromagnetic, magnetic, or electric signal) can be coated onto a device or a portion of a device. In another example, a coating can be utilized to carry a payload/agent. In certain embodiments, an agent can be associated with individual layers of a multilayer coating for incorporation, affording an opportunity for exquisite control of loading and release from the coating. For instance, an agent can be incorporated into a multilayer coating by serving as a layer.

In some embodiments, a coating comprises a targeting material such as antibodies, aptamers). Such coatings or materials can be used in combination with any other coating disclosed therein.

In some embodiments, a coating comprises an adhesive material as discussed above. For example, a coating can comprise a bioadhesive such as chitosan, carbopol, catechol group-modified polymers, and catechol group-modified hydrogels. Such coatings or materials can be used in combination with any other coating disclosed therein.

### Method of Use

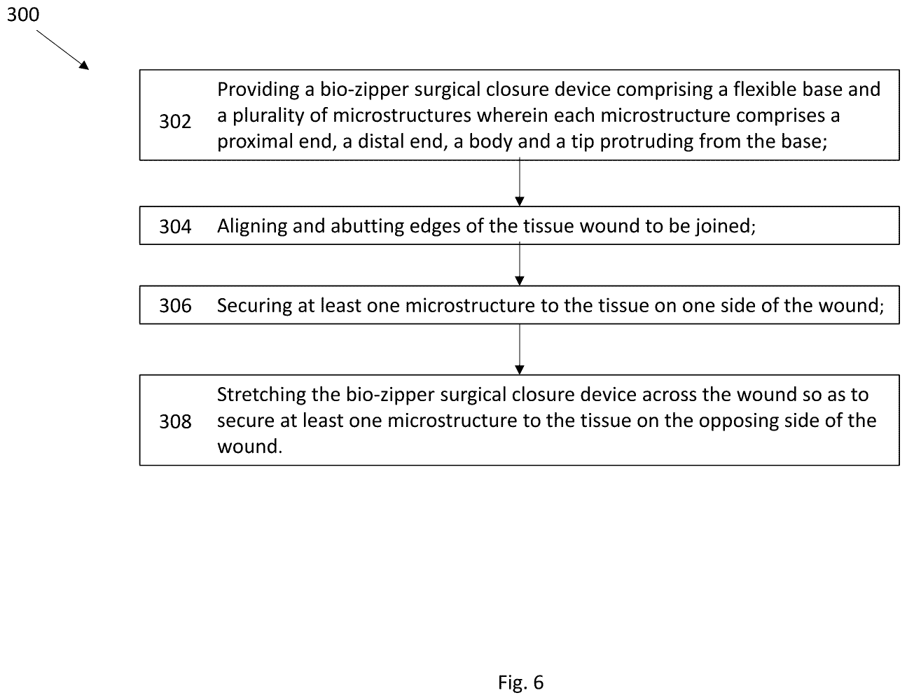

The present invention also relates to methods for the closure of various wounds. Referring now to FIG. 6, an exemplary method 300 of wound closure using the bio-zipper surgical closure device 100 is depicted. Method 300 begins with step 302, wherein a bio-zipper surgical closure device is provided, the bio-zipper surgical closure device comprising a flexible base and a plurality of microstructures, each microstructure comprising a proximal end, a distal end, a body and a tip protruding from the base. In step 304, abutting edges of a tissue wound to be joined are aligned adjacent to each other. In step 306, at least a part of the adhesive layer is secured to the tissue on one side of the wound. In step 308, the bio-zipper surgical closure device is stretched across the wound so as to secure at least a part of the adhesive layer to the tissue on the opposing side of the wound.

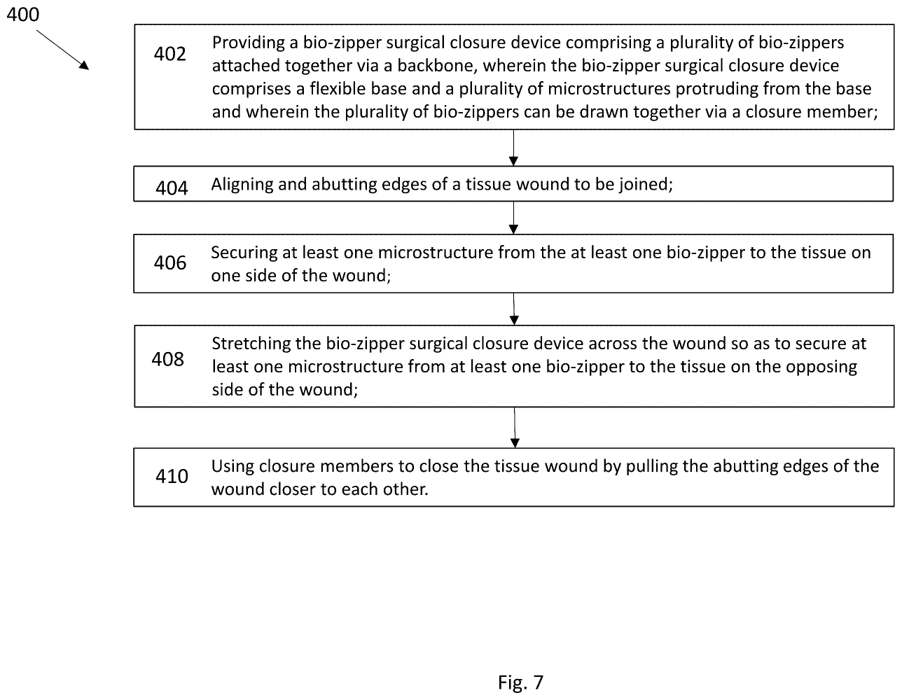

Referring now to FIG. 7, another exemplary method 400 of wound closure using the bio-zipper surgical closure device 100 is depicted. Method 400 begins with step 402, wherein a plurality of bio-zipper surgical closure devices attached together via a backbone is provided, the bio-zipper surgical closure device comprising a flexible base and a plurality of microstructures protruding from the base. In step 404, abutting edges of a tissue wound to be joined are aligned adjacent to each other. In step 406, at least a part of the adhesive layer from the at least one bio-zipper is secured to the tissue on one side of the wound. In step 408, the bio-zipper surgical closure device is stretched across the wound so as to secure at least a part of the adhesive layer from the at least one bio-zipper to the tissue on the opposing side of the wound. In step 410, closure members are used to close the tissue wound by pulling the abutting edges of the wound closer to each other.

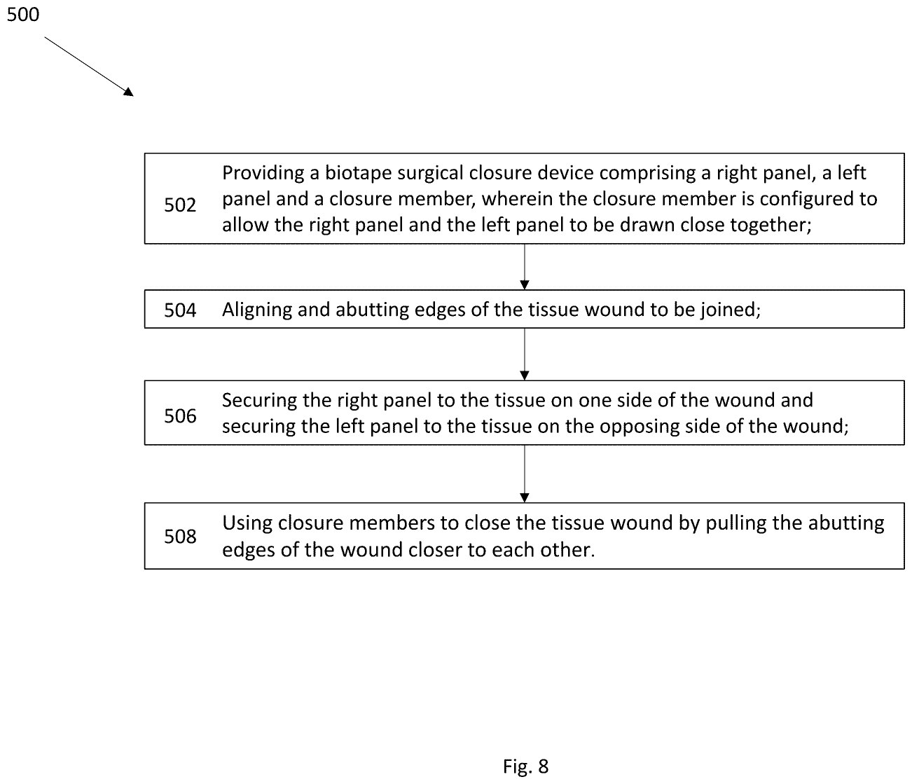

Referring now to FIG. 8, another exemplary method 500 of wound closure using the biotape surgical closure device 200 is depicted. Method 500 begins with step 502, wherein a biotape surgical closure device comprising a right panel, a left panel and a closure member is provided. In step 504, abutting edges of a tissue wound to be joined are aligned adjacent to each other. In step 506, the right panel is secured to the tissue on one side of the wound and the left panel is secured to the tissue on the opposing side of the wound. In step 508, closure members are used to close the tissue wound by pulling the abutting edges of the wound closer to each other.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### Example 1: Development of a Biodegradable Adhesive Polymer

Given the need for biodegradable adhesives, modified biodegradable polymers were chosen for investigation. In order to achieve a suitable adhesive polymer, it must strongly adhere to soft tissue within seconds of application. Additionally, the adhesive cannot be loosened by moisture, blood, or urine and must remain strong in physiological conditions of stretching and compressing, typically for a minimum of four weeks. If the adhesive is being used to adhere additional material to a tissue, it cannot delaminate from the material. Most importantly, the adhesive must not be toxic or promote cellular death in the tissue it has been applied to. For this reason, poly(glycerin sebacate) PGS was selected as the base for initial studies of adhesive biocompatible, biodegradable polymers.

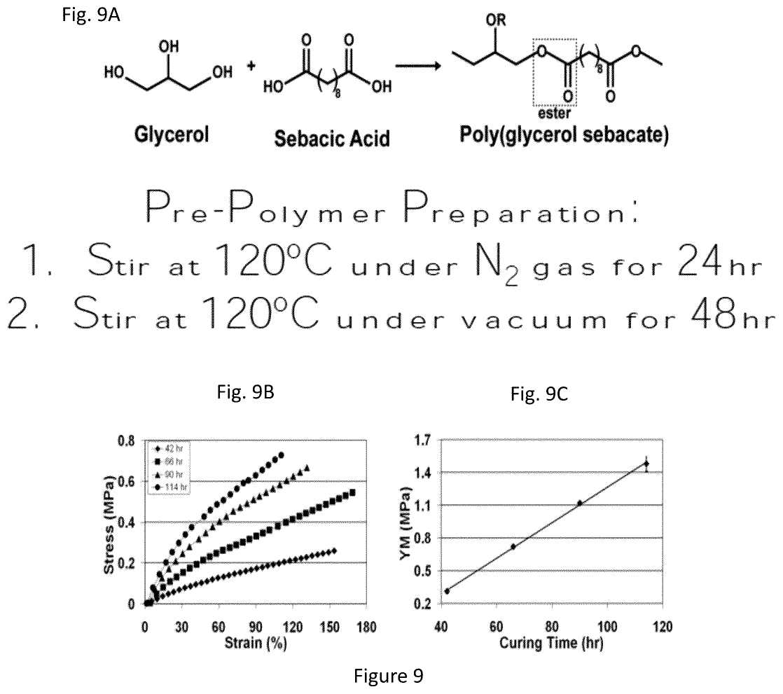

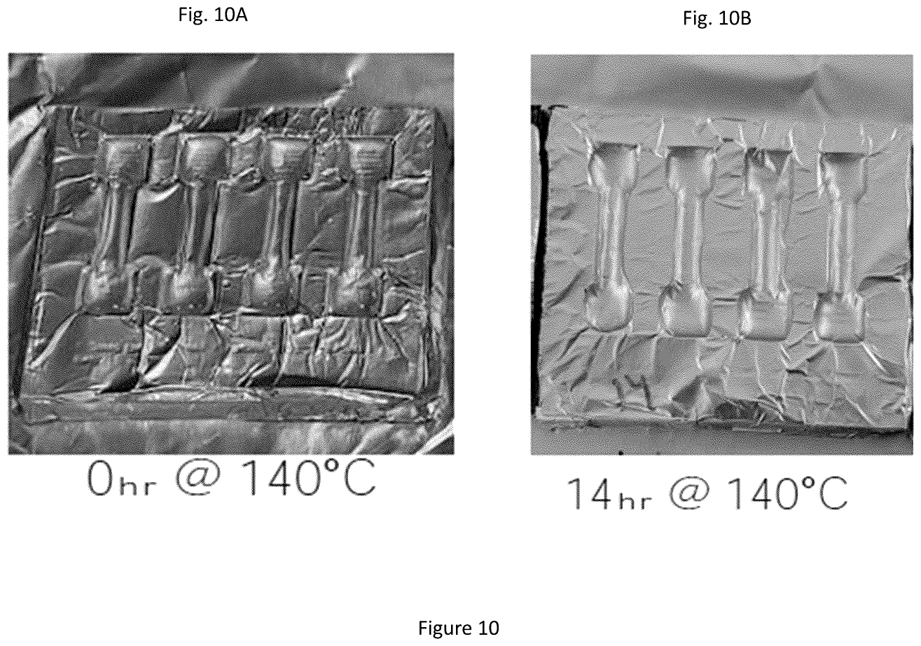

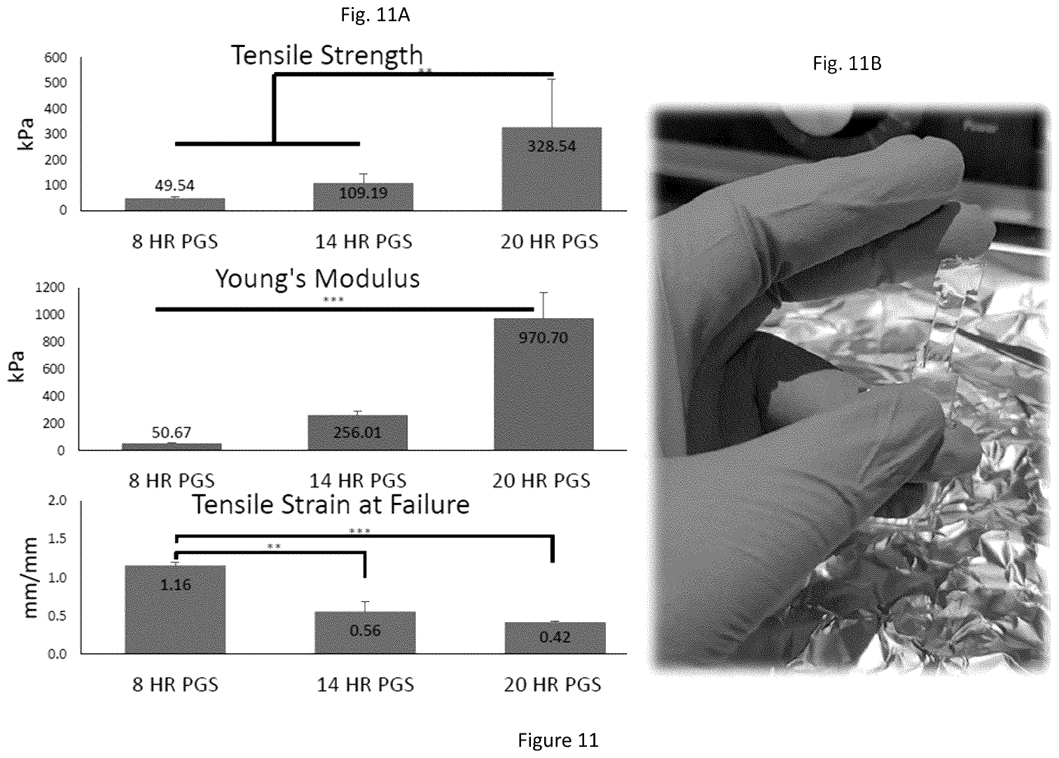

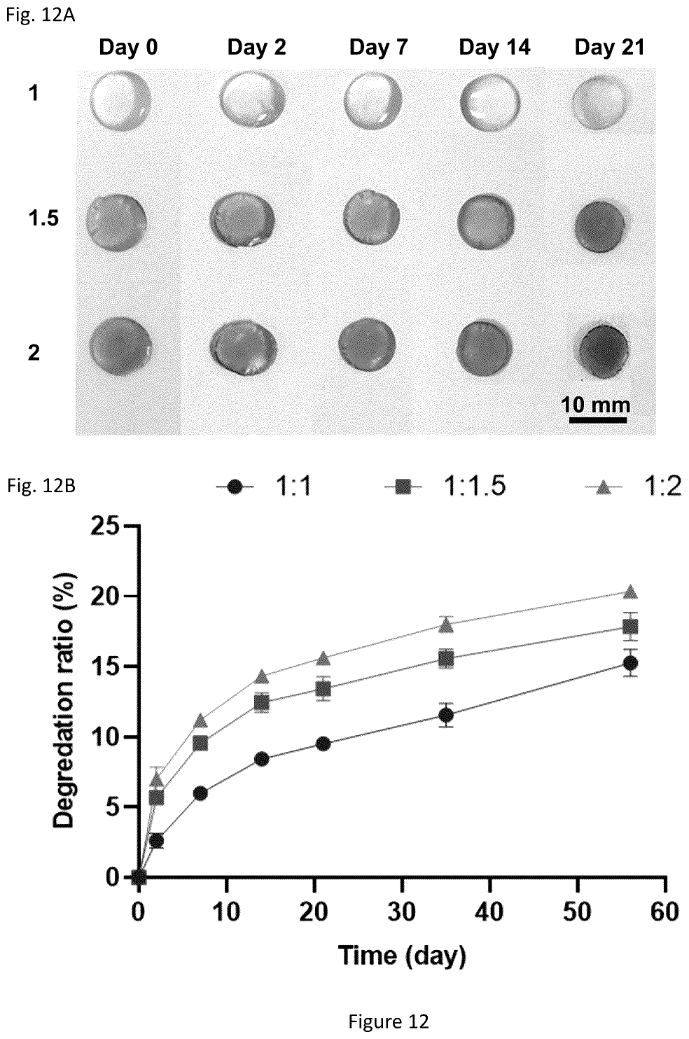

Unmodified PGS was prepared by stirring glycerol and sebacic acid under N₂ at 120° C. for 24 hours and then stirring under vacuum at 120° C. for 48 h (Pomerantseva I. et al., 2009, Journal of Biomedical Materials Research Part A, 91A: 1038-1047; Wang Y. et al., 2002, Nature Biotech., 20:602-606) (FIG. 9A). Secondary curing of the PGS polymer was subsequently performed by heating prepolymer above 100° C. and pouring the prepolymer into non-stick aluminum foil over laser etched poly(methyl methacrylate) (PMMA) mold. The prepolymer was degassed under vacuum at 120° C. for one hour, polymerized at 140° C., removed from mold, and used in trials (FIG. 10A and FIG. 10B). The tunable range of elastomer strengths of PGS bars were subsequently evaluated (FIG. 11A and FIG. 11B). Investigation of PGS for biodegradation demonstrated that the polymer, while biodegradable, does so at an reasonable rate, with less than 15% degradation over 20 days of incubation in human plasma (FIG. 12).

For modification of PGS, L-DOPA was selected because of the strong adhesion in wet environments that has been linked to the catechol-containing amino acid L-DOPA (Mehdizadeh M., 2012, Biomaterials, 33:7972-7983) and formation of covalent bonds with available nucleophile groups on these surfaces, such as —NH₂, —SH, —OH, and —COOH groups (Mehdizadeh M., 2012, Biomaterials, 33:7972-7983).

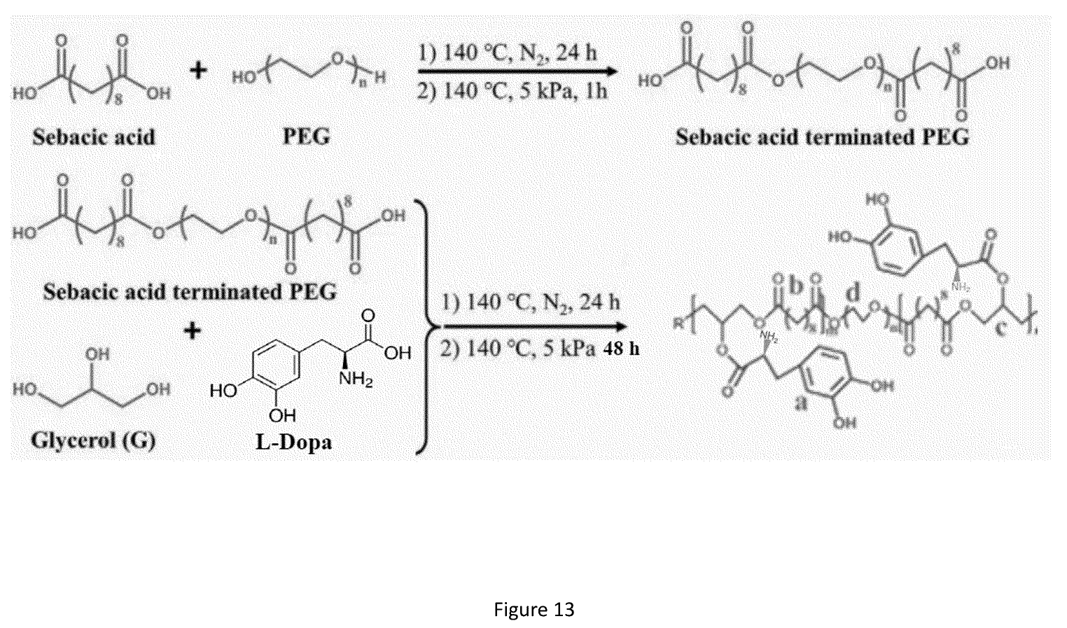

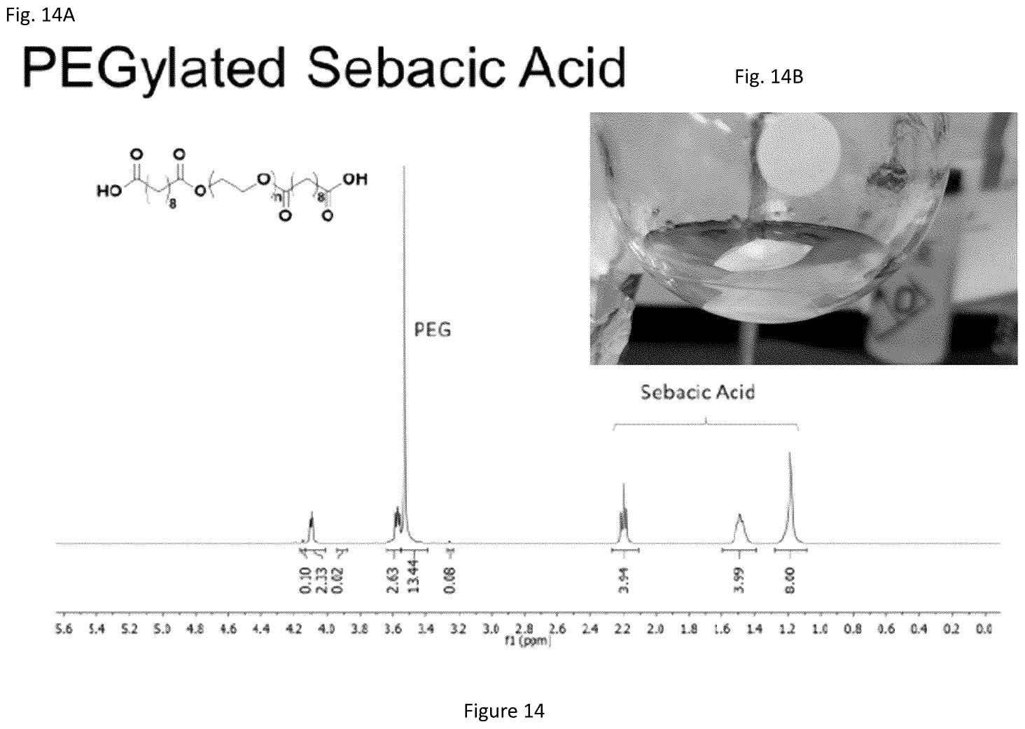

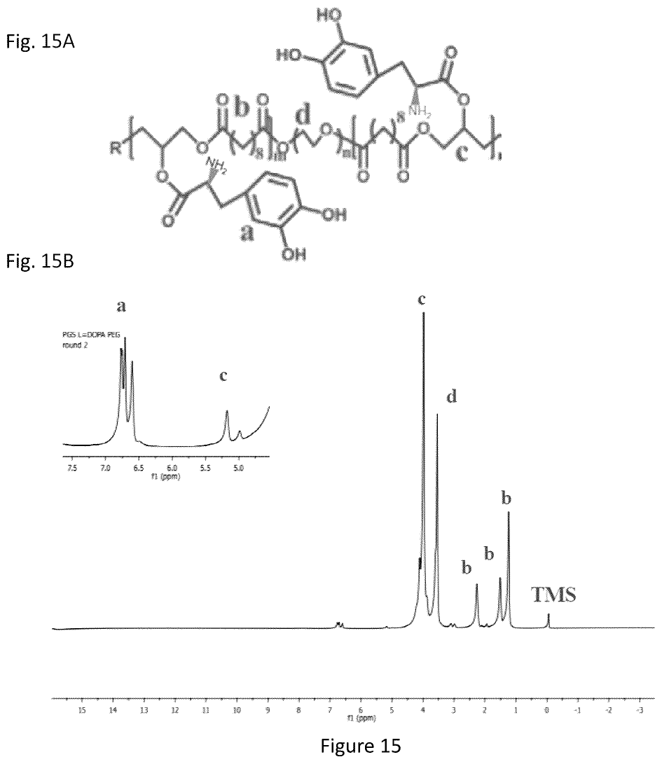

Sebacic acid-PEG was prepared similarly to PGS. Sebacic acid and PEG 400 or PEG1000 were stirred under N₂ at 140° C. for 24 hours, after which it was incubated under vacuum at 140° C. for one hour (FIG. 13), and successful coupling was confirmed by ¹H NMR (FIG. 14A and FIG. 14B). The Sebacic acid-PEG (SA-PEG) was then stirred with glycerol and L-3,4-dihydroxyphenylalanine (L-DOPA) at 140° C. for 24 hours. ¹H NMR of PGS-PEG-L-DOPA showed the characteristic proton NMR peaks at 6.5-6.7 ppm of phenyl group protons from L-DOPA, peaks from methylene (—CH₂—) protons of sebacic acid at 1.2, 1.5, and 2.3 ppm, peaks from glycerol protons at 4.0-4.2 ppm and 4.9-5.2 ppm, and peaks from methylene (—CH₂—) protons of PEG at 3.5 ppm (FIG. 15A and FIG. 15B).

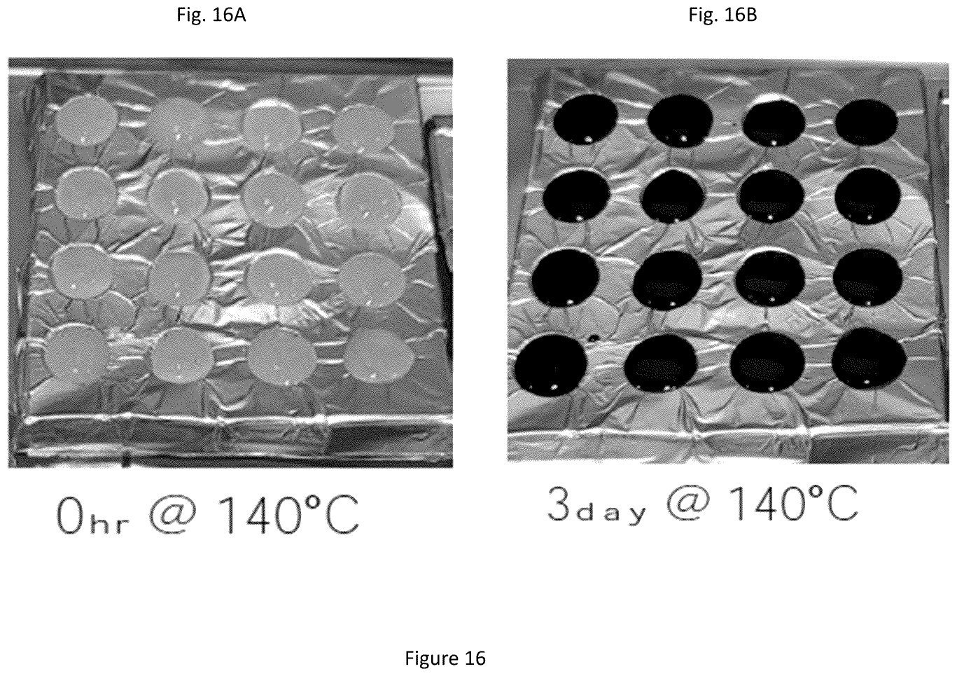

Secondary curing PGS-PEG-L-DOPA polymer was subsequently prepared by heating prepolymer above 100° C. and pouring the prepolymer into non-stick aluminum foil over laser etched polydimethylsiloxane (PDMS) mold. The prepolymer was degassed and polymerized under vacuum at 140° C. for up to 120 hours (FIG. 16A and FIG. 16B).

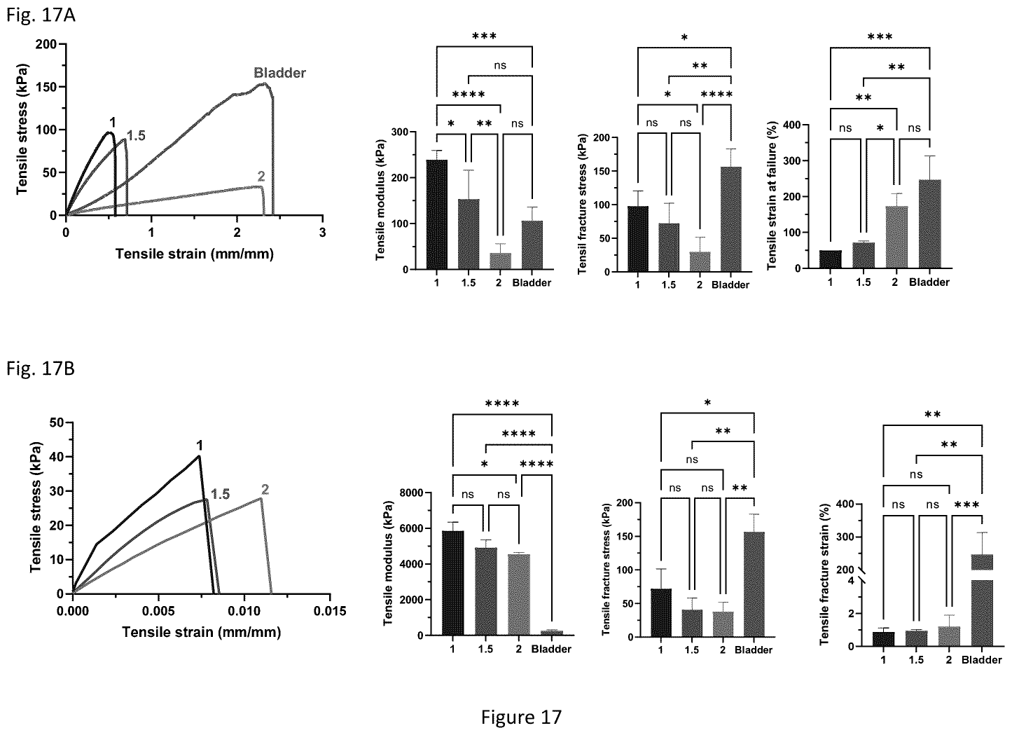

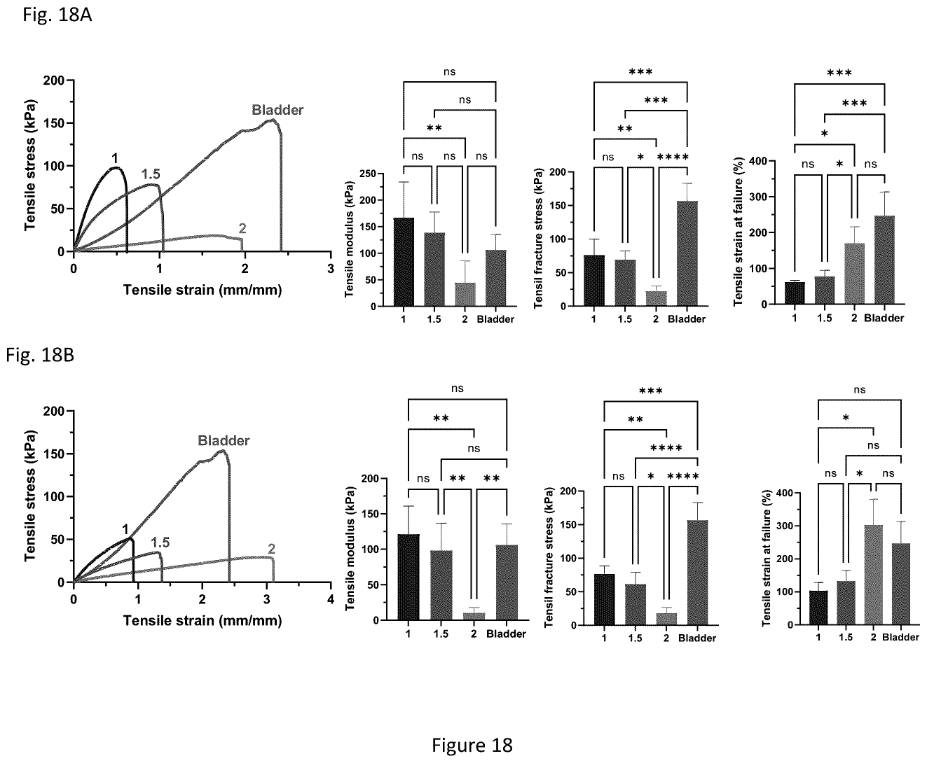

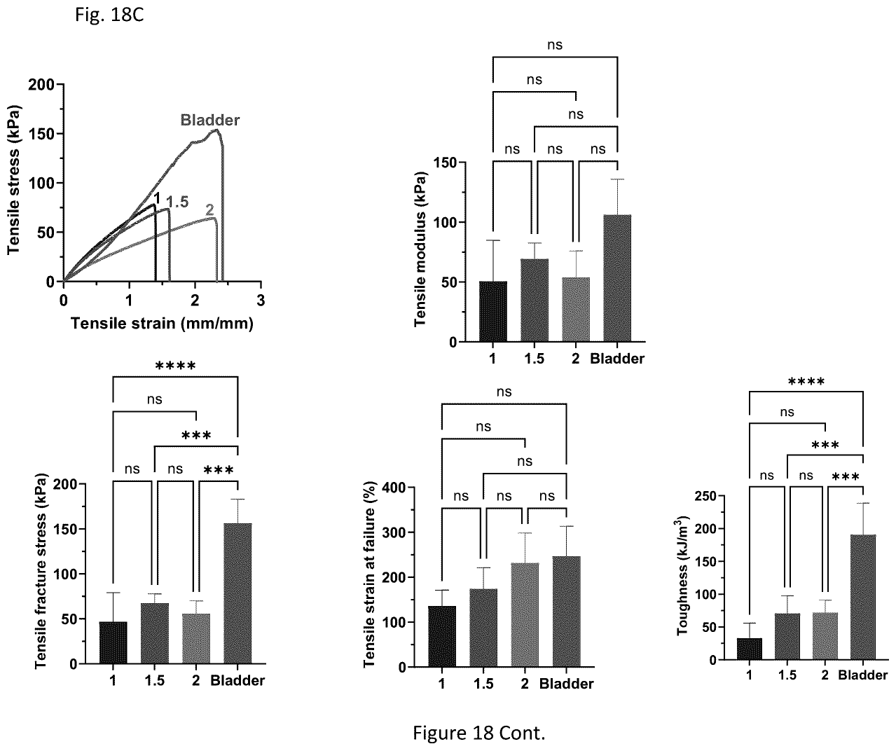

The mechanical properties of the adhesive polymer were next investigated, where it was determined that increasing the ratio of glycerol increases the elasticity of PGS derivatives (FIG. 17A). In examining the effect of the PEG utilized, it was observed that high molecular weight PEG (PEG 3000) results in brittle polymer (FIG. 17B). Incorporation of L-DOPA and PEG 400 successfully produced polymers with the desired mechanical properties (FIG. 18A through FIG. 18C).

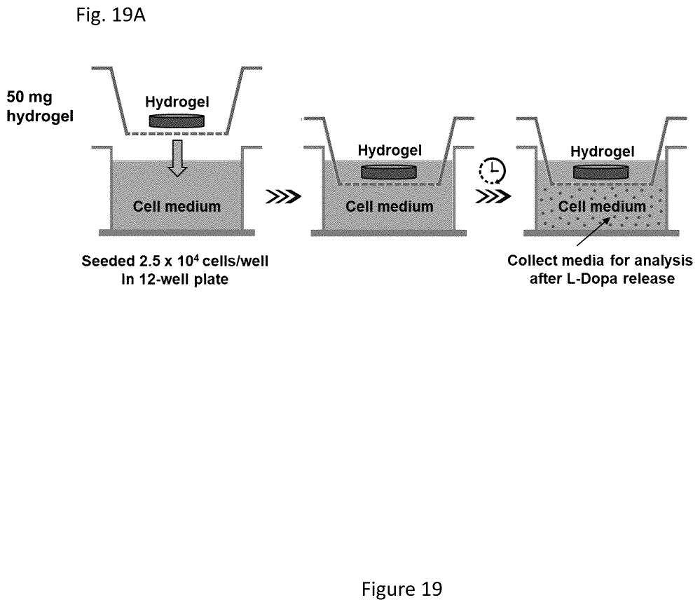

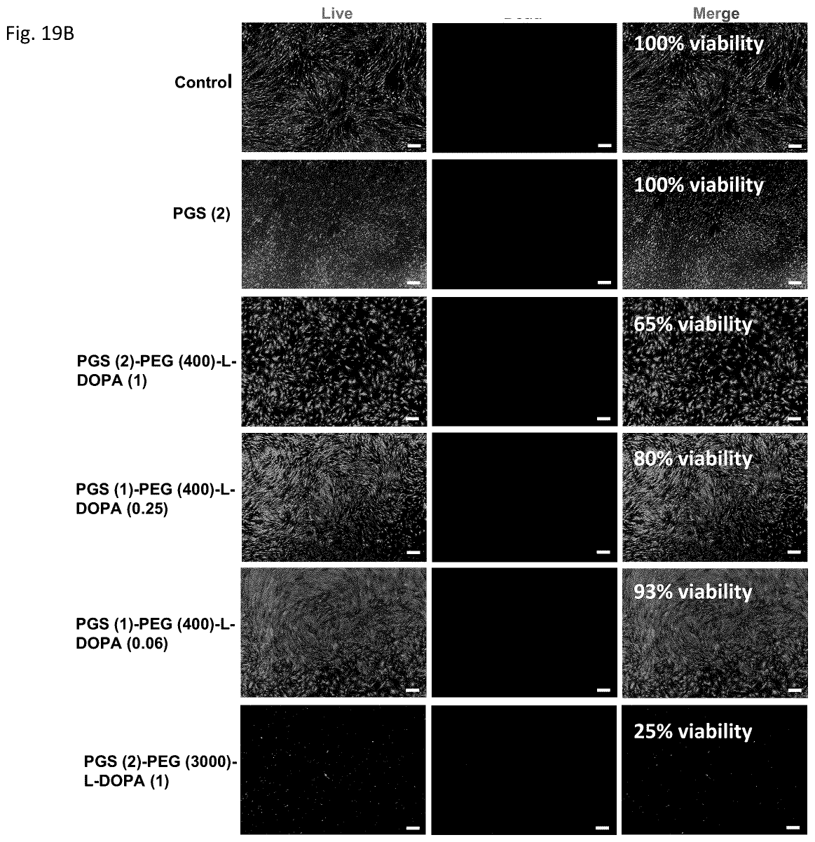

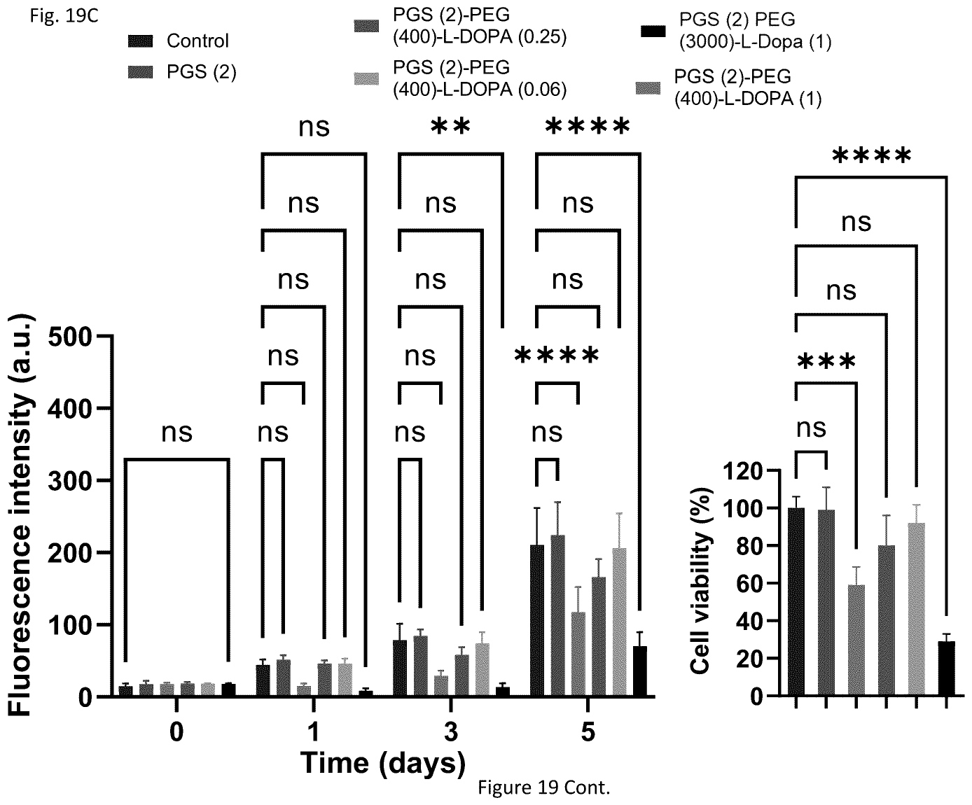

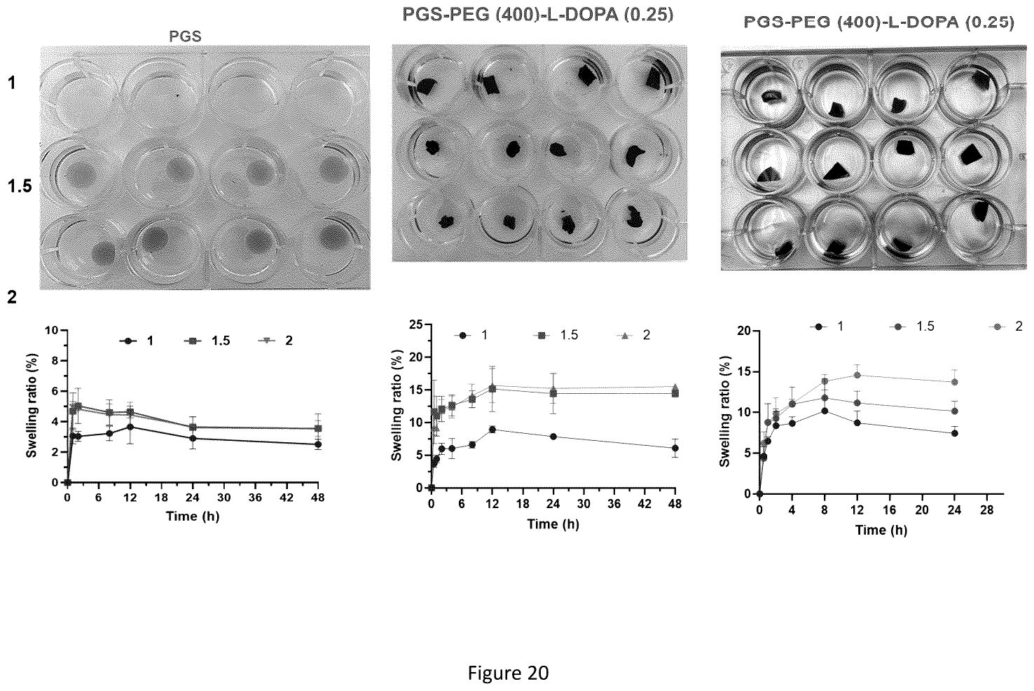

Examination of the cytotoxicity of the polymers, however, demonstrated that high-L-DOPA content resulted in reduced cell viability (FIG. 19). Accordingly, polymers with 0.06 or 0.25 L-DOPA loading were used for further study. In comparing traditional PGS with PGS-PEG-L-DOPA, it was found that, while swelling of polymer was approximately 3-fold greater than traditional PGS, the swelling was within acceptable clinical ranges (FIG. 20).

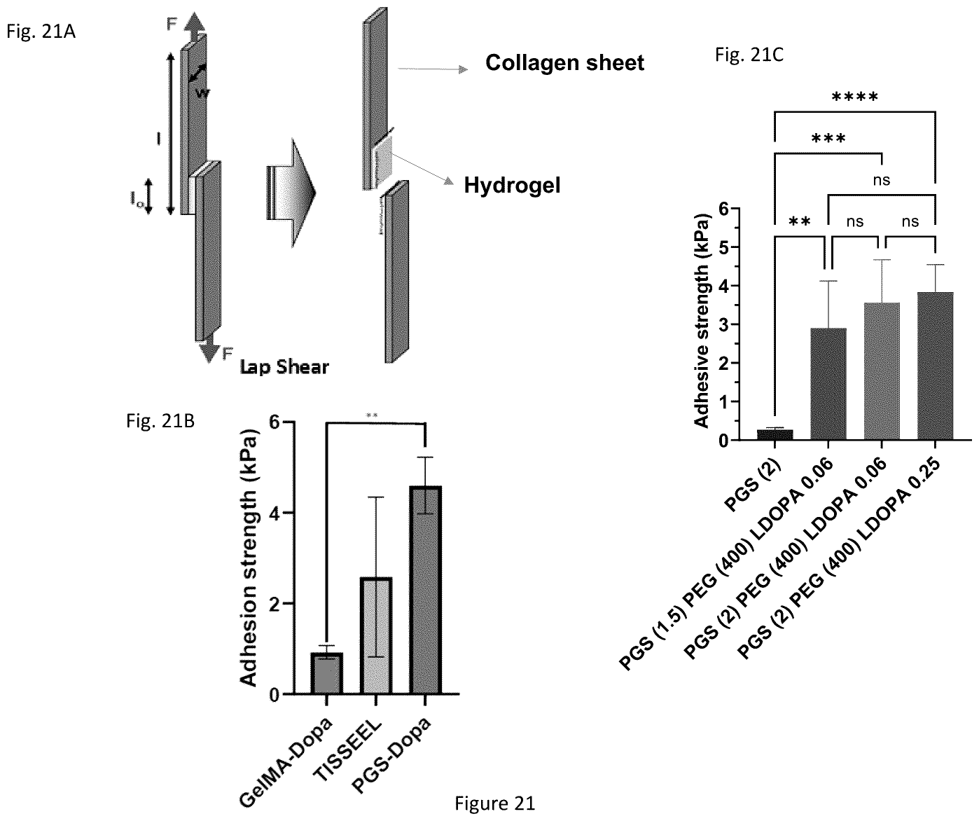

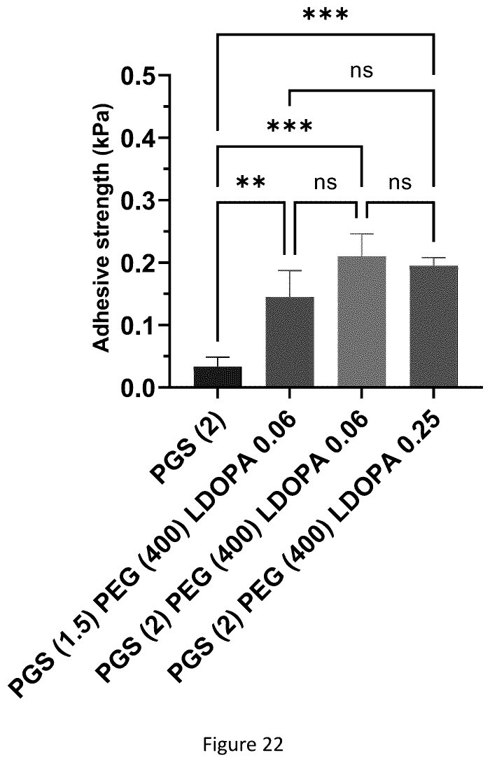

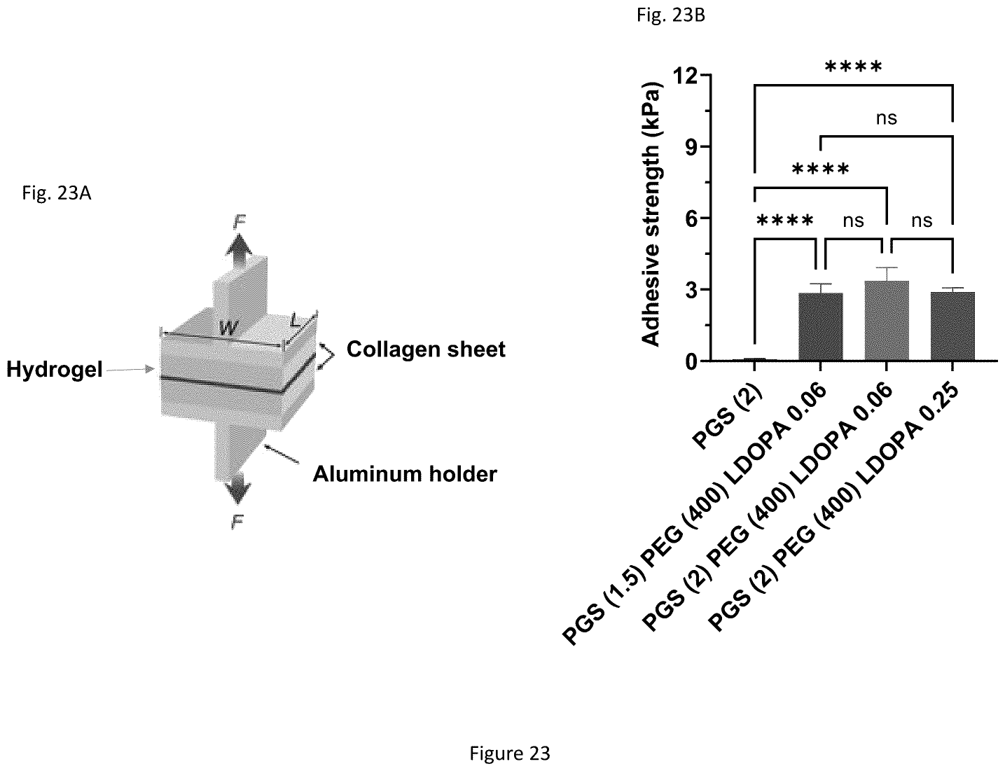

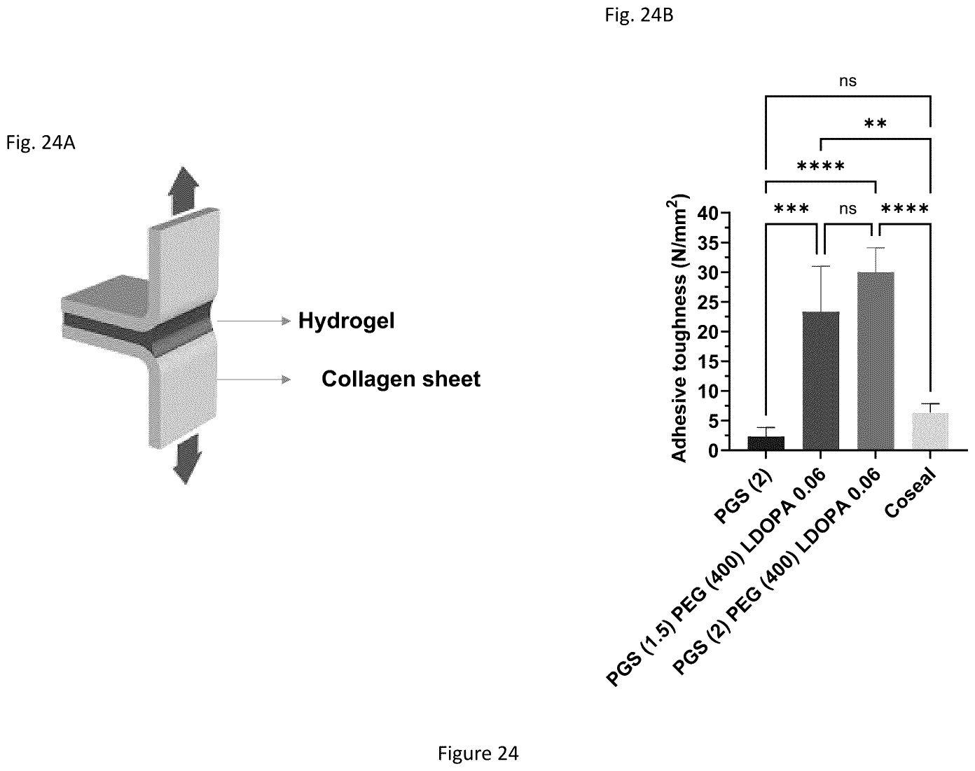

The adhesive properties of the polymer were next examined in three different assays. Lap shear assays were performed on collagen sheets (FIG. 21A and FIG. 21B) and bladder tissue (FIG. 22), where PGS-PEG-L-DOPA demonstrated superior adhesion strength. Examination of tensile adhesive strength on collagen sheets further demonstrated successful adhesion (FIG. 23). The adhesive strength was also evaluated in a peeling test on collagen sheets (FIG. 24).

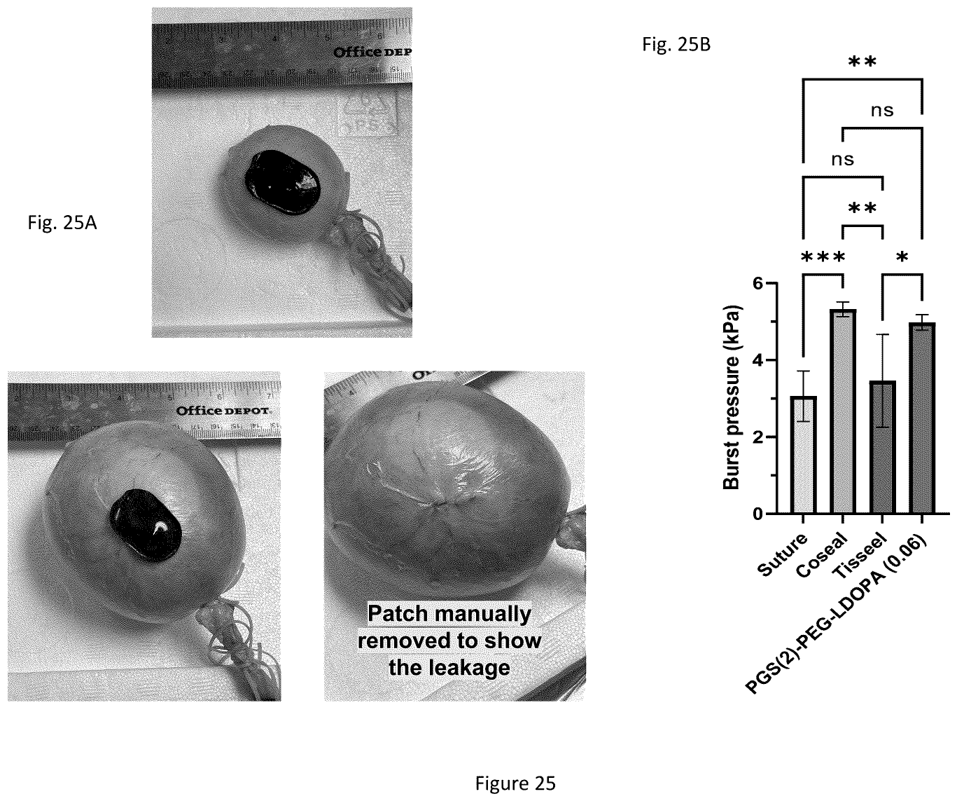

As PGS-PEG-L-DOPA demonstrated impressive adhesive strength, its ability to improve support healing of a surgical closure was examined. A porcine bladder was punctured with a 1 cm incision, which was closed with a double-layer stitch closure with 3-0 VICRYL. The closure was then tested by pumping dyed water into the bladder while monitoring for a leak, with the pressure recorded upon leak or a sudden pressure drop. Physiological bladder voiding pressure was demonstrated to be between 2.0 to 7.8 kPa (Lim C. S., 1995, World J. Urol., 13:34-39). The assay was performed with only the suture or with the suture coupled with a Coseal patch, a Tisseel patch, or a PGS-PEG-L-DOPA patch. The modified PGS patch yielded results superior to the suture with Tisseel and comparable to Coseal (FIG. 25).

### Example 2: Use of an Adhesive Polymer in a Closure Device

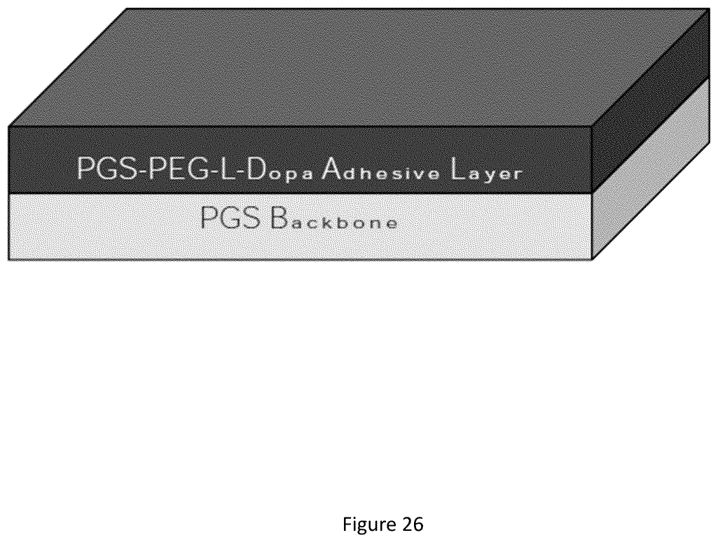

Given PGS-PEG-L-DOPA's superior adhesive properties, a 2-layer patch was formed on a PGS backbone (FIG. 26). More specifically, PGS/PGS-PEG-L-DOPA 2-layer patch was prepared by curing PGS and PGS-PEG-L-DOPA prepolymer in sequence on top of each other, where PGS was cured in mold at 140° C. for 24 hours, PGS-PEG-L-DOPA prepolymer was then added on top, and the system was cured for additional 19 hours, to form the desired polymerized patch.

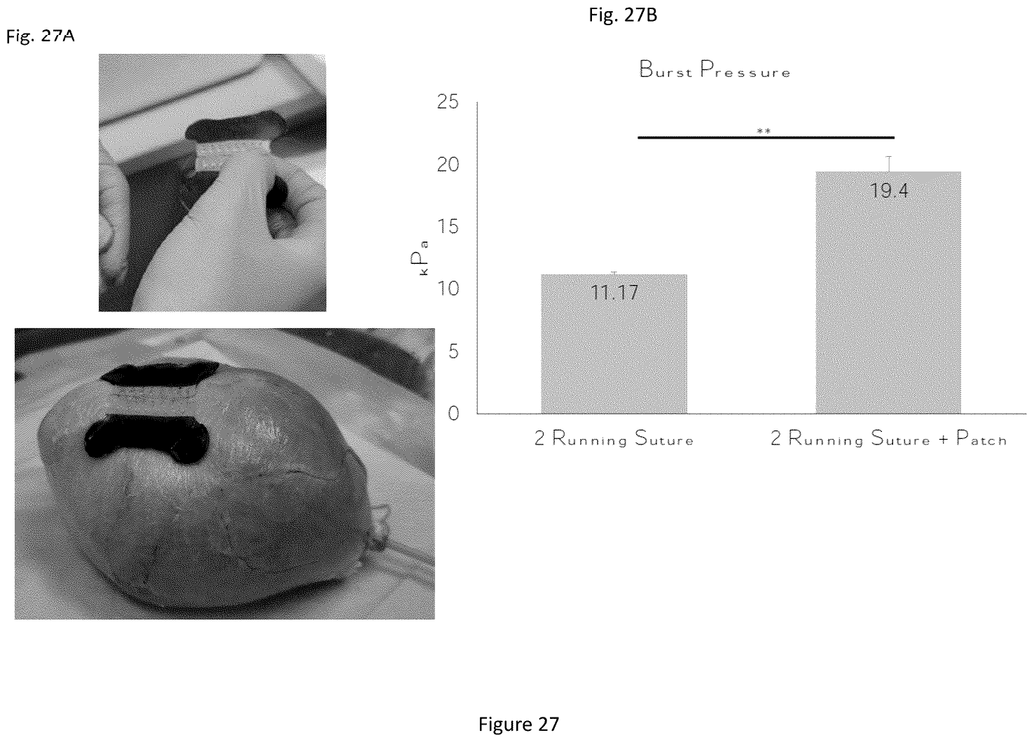

Subsequent bladder burst pressure testing, as previously performed with a PGS-PEG-L-DOPA-only patch were performed by cutting 1-2 cm defect into Porcine bladder. The defect was sutured shut with two running sutures or two running sutures and a PGS/PGS-PEG-L-DOPA patch. Dyed water was pumped into closed bladder while pressure was monitored and recorded when leak appeared or a sudden pressure drop was observed (FIG. 27A). As shown in FIG. 27B, functionalized PGS increased the pressure required for the wound closure to leak. The burst pressure of the porcine bladder incision that was closed with two running sutures was recorded at 11.17 kPa, while the burst pressure of porcine bladder incision that was closed with two running sutures and the adhesive patch was significantly higher and was recorded at 19.4 kPa demonstrating the superior properties of PGS/PGS-PEG-L-DOPA patch.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An adhesive composition comprising the reaction product of:
a) a linear dicarboxylic acid;
b) a saturated triol; and
c) an aromatic amino acid;
wherein the adhesive is non-cytotoxic and biodegradable.

2. The adhesive composition of claim 1, wherein the linear dicarboxylic acid comprises the reaction product of a saturated linear dicarboxylic acid and a linear polymer.

3. The adhesive composition of claim 2, wherein the saturated linear dicarboxylic acid is one or more selected from the group consisting of: succinic acid, glutaric acid, adipic acid, pimelic acid, suberic acid, azelaic acid, sebacic acid, undecanedioic acid, dodecanedioic acid, brassylic acid, tetradecanedioic acid, pentadecanedioic acid, thapsic acid, heptadecanedioic acid, japanic acid, and phellognic acid.

4. The adhesive composition of claim 1, wherein the linear dicarboxylic acid is one or more selected from the group consisting of: succinic acid, glutaric acid, adipic acid, pimelic acid, suberic acid, azelaic acid, sebacic acid, undecanedioic acid, dodecanedioic acid, brassylic acid, tetradecanedioic acid, pentadecanedioic acid, thapsic acid, heptadecanedioic acid, japanic acid, and phellognic acid.

5. The adhesive composition of claim 2, wherein the linear polymer is one or more selected from the group consisting of poly(ethylene glycol) (PEG), poly(lactic acid) (PLA), poly(caprolactone) (PCL), poly(glycolic acid) (PGA), poly(lactic-co-glycolic acid) (PLGA), and poly(glycerol sebacate) (PGS).

6. The adhesive composition of claim 4, wherein the linear polymer is PEG, wherein the PEG comprises one or more selected from the group consisting of: PEG2, PEG3, PEG4, PEG5, PEG6, PEG7, PEG8, PEG9, or PEG10.

7. The adhesive composition of claim 1, wherein the saturated triol is one or more selected from the group consisting of glycerol, 1,2,4-butanetriol, 2-hydroxymethyl-1,3-propanediol, 1,3,5-propanetriol, 2-hydroxymethyl-1,4-butanediol, 1,2,6-hexanetriol, 1,3,6-hexanetriol, 2-hydroxymethyl-1,5-pentanediol, 3-hydroxymethyl-1,5-pentanediol, 1,2,7-heptanetriol, 1,3,7-heptanetriol, 1,4,7-heptanetriol, 2-hydroxymethyl-1,6-hexanediol, 3-hydroxymethyl-1,6-hexanediol, and 3-hydroxyethyl-1,5-pentanediol.

8. The adhesive composition of claim 1, wherein the aromatic amino acid is of formula (I): 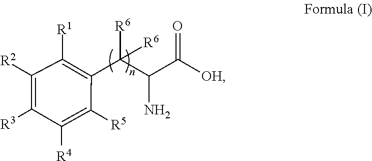
wherein R¹-R⁵ are each independently selected from the group consisting of H, D, F, Cl, Br, hydroxyl, hydroxymethyl, hydroxyethyl, methoxy, ethoxy, methyl, ethyl, amino, aminomethyl, aminoethyl, methylamino, and ethylamino;
wherein at least one of R¹-R⁵ is a hydroxyl;
wherein n=1-6; and
wherein each instance of R⁶ is independently selected from H, D, F, and Cl.

9. The composition of claim 1, wherein the reaction product of a saturated linear dicarboxylic acid and a linear polymer comprises 40-60% saturated linear dicarboxylic acid by weight.

10. The composition of claim 1, wherein the composition comprises 50-85% reaction product of a saturated linear dicarboxylic acid and a PEG by weight.

11. The composition of claim 1, wherein the composition comprises 10-30% saturated triol by weight.

12. The composition of claim 1, wherein the composition comprises 1-5% aromatic amino acid by weight.

13. The composition of claim 1, wherein the saturated linear dicarboxylic acid is sebacic acid, the PEG is PEG400, the saturated triol is glycerol, and the aromatic amino acid is L-DOPA.

14. The composition of claim 12, wherein the composition comprises: 50-85% reaction product of sebacic acid and PEG400 by weight, 10-30% glycerol by weight, and 1-5% L-DOPA by weight;
wherein the reaction product of sebacic acid and PEG400 is 40-60% saturated linear carboxylic acid by weight.

15. A self-adhesive device comprising:
a flexible base; and
an adhesive layer comprising an adhesive according to any one of claims 1-13;
wherein the flexible base is non-toxic.

16. The self-adhesive device of claim 15, wherein the flexible base comprises a biodegradable polymer.

17. (canceled)

18. The self-adhesive device of claim 16, wherein the biodegradable polymer is selected from the group consisting of polylactic acid (PLA), poly(L-lactic acid) (PLLA), poly(D,L-lactic acid) (PDLLA), polycaprolactone (PCL), chitosan, polyglycolic acid (PGA), poly(lactic-go-glycolic acid) (PLGA), poly(3-hydroxybuterate-co-3-hydroxyvalerate (PHBV), chitin, polyhydroxyalkanoates (PHAs), polyamides, poly(glycerol sebacate) (PGS), polydioxanone (PDS), poly(trimethylene carbonate) (PTMC), cyanophycin, PEG, polyethylene glycol diacrylate (PEGDA), poly(vinyl alcohol) (PVA), poly(lactic acid-co-caprlocactone) (PLCL), poly(ester urea) (PEU), and poly(ester urethane).

19. The self-adhesive device of claim 17, wherein the polymer is PGS.

20. The self-adhesive device of claim 16, wherein the device is a biozipper or a biotape.

21. (canceled)

22. The self-adhesive device of claim 16, wherein the adhesive layer comprises a therapeutic agent.
